(19) United States
(12) Patent Application Publication
(10) Pub. No.: US 2009/0081177 A1
(43) Pub. Date: Mar. 26, 2009

(54) Methods and compositions for improving pregnancy outcome

(75) Inventor: Kelton Paul Tremellen, South Australia (AU)

Correspondence Address: HOWSON & HOWSON LLP, 501 OFFICE CENTER DRIVE, SUITE 210, FORT WASHINGTON, PA, 19034, US

(73) Assignee: FERTILITY TECHNOLOGIES PTY LTD, ADELAIDE (AU)

(21) Appl. No.: 11/919,726

(22) PCT Filed: Jul. 5, 2006
(86) PCT No.: PCT/AU2006/000939

§ 371 Date: Oct. 31, 2007

Related U.S. Application Data
(60) Provisional application No. 60/696,746, filed on Jul. 5, 2005.

(51) Int. Cl. A61K 31/202; A61P 15/00; A61K 31/375; A61K 31/355; A61K 31/122; A61K 33/04; A61K 31/522; A61K 36/8962; A01K 67/00; C12N 5/08; A61K 39/395; A61K 36/906; A61K 33/30; A61K 31/19; A61K 31/22; A61K 36/82

(52) U.S. Cl. 424/94.1; 514/474; 514/458; 424/702; 424/641; 514/561; 514/547; 514/562; 424/729; 514/560; 424/756; 514/263.31; 424/133.1; 424/754; 435/366; 800/8

(57) ABSTRACT

A method of increasing pregnancy rate in a female subject is provided, the female subject or an oocyte for introduction into the female subject being fertilized by a sperm from a male subject. The method includes the steps of administering to the male subject prior to fertilization: (i) an effective amount of an anti-oxidant agent; and (ii) an effective amount of an agent that reduces inflammation in the male reproductive tract and/or an effective amount of an agent that increases testicular testosterone concentration.

This application claims priority from U.S. Provisional Patent Application No. 60/696,746 filed on 5 Jul. 2005, the contents of which are to be taken as incorporated herein by this reference.

### FIELD OF THE INVENTION

The present invention relates to methods and compositions for improving pregnancy rate and improving pregnancy outcome.

The present invention also relates to methods and compositions for reducing damage to sperm in a male subject.

### BACKGROUND OF THE INVENTION

Despite numerous advances in reproductive medicine, a significant proportion of couples are unable to conceive due to reduced male fertility. Although there are numerous causes for reduced male fertility, free radical damage to spermatozoa appears to be one of the primary factors contributing to reduced fertility. High levels of free radicals have been measured in semen of men with infertility of unknown origin, smokers, infertility related to poor sperm motility and in men following vasectomy reversal.

Free radicals are capable of interfering with fertility by one of three mechanisms. Firstly, free radical damage to the sperm membrane interferes with function of the sperm tail, reducing sperm motility. Secondly, free radical damage to the headpiece membrane can interfere with the acrosome reaction, a natural response vital to oocyte-sperm fusion and fertilization. Finally, if semen free radical levels are very high they can damage the sperm genetic material (DNA) leading to poor embryo quality and infertility/miscarriage. Sperm DNA damage caused by the father smoking has also been linked to the development of childhood cancers in their progeny in a number of studies.

The use of assisted reproduction techniques has allowed intervention to treat poor fertility. However, despite the numerous advances made in the field of assisted reproduction, the rate of success of assisted reproduction techniques still remains generally low. The low rates of success of assisted reproduction techniques such as in vitro fertilization (IVF), intracytoplasmic sperm injection (ICSI) and intra-uterine insemination (IUI) is likely to be due in part to free radical damage to sperm that occurs endogenously in the male.

Accordingly, there is a need for new methods and compositions to improve pregnancy rate and outcome for natural and assisted pregnancies. The present invention relates to methods and compositions for improving pregnancy rate and outcome in assisted and natural pregnancies, and to methods and compositions for reducing the damage to sperm produced in a male subject, by administering to the male an anti-oxidant in combination with an agent that reduces inflammation in the male reproductive tract and/or an agent that increases testicular testosterone concentration.

A reference herein to a patent document or other matter which is given as prior art is not to be taken as an admission that that document or matter was known or that the information it contains was part of the common general knowledge as at the priority date of any of the claims.

### SUMMARY OF THE INVENTION

The present invention provides a method of increasing pregnancy rate in a female subject, the female subject or an oocyte introduced into the female subject being fertilized by a sperm from a male subject, the method including the steps of administering to the male subject prior to fertilization:
(i) an effective amount of an anti-oxidant agent; and
(ii) an effective amount of an agent that reduces inflammation in the male reproductive tract and/or an effective amount of an agent that increases testicular testosterone concentration.

The present invention also provides a method of improving pregnancy outcome in a female subject, the female subject or an oocyte for introduction into the female subject fertilized by a sperm from a male subject, the method including the steps of administering to the male subject prior to fertilization:
(i) an effective amount of an anti-oxidant agent; and
(ii) an effective amount of an agent that reduces inflammation in the male reproductive tract and/or an effective amount of an agent that increases testicular testosterone concentration.

The present invention also provides a method of reducing free radical damage to sperm produced by a male subject, the method including the steps of administering to the male subject:
(i) an effective amount of an anti-oxidant agent; and
(ii) an effective amount of an agent that reduces inflammation in the male reproductive tract and/or an effective amount of an agent that increases testicular testosterone concentration.

The present invention also provides a method of reducing generation of free radicals in the reproductive tract and/or in semen of a male subject, the method including the steps of administering to the male subject:
(i) an effective amount of an anti-oxidant agent; and
(ii) an effective amount of an agent that reduces inflammation in the male reproductive tract and/or an effective amount of an agent that increases testicular testosterone concentration.

The present invention also provides a method of reducing activity and/or concentration of leukocytes in the reproductive tract and/or semen of a male subject, the method including the steps of administering to the male subject:
(i) an effective amount of an anti-oxidant agent; and
(ii) an effective amount of an agent that reduces inflammation in the male reproductive tract and/or an effective amount of an agent that increases testicular testosterone concentration.

The present invention also provides a method of reducing the level and/or production of one or more inflammatory agents in the reproductive tract and/or semen of a male subject, the method including the steps of administering to the male subject:
(i) an effective amount of an anti-oxidant agent; and
(ii) an effective amount of an agent that reduces inflammation in the male reproductive tract and/or an effective amount of an agent that increases testicular testosterone concentration.

The present invention also provides a method of improving sperm function in a male subject, the method including the steps of administering to the male subject:
(i) an effective amount of an anti-oxidant agent; and
(ii) an effective amount of an agent that reduces inflammation in the male reproductive tract and/or an effective amount of an agent that increases testicular testosterone concentration.

The present invention also provides a method of improving sperm motility in a male subject, the method including the steps of administering to the male subject:
(i) an effective amount of an anti-oxidant agent; and
(ii) an effective amount of an agent that reduces inflammation in the male reproductive tract and/or an effective amount of an agent that increases testicular testosterone concentration.

The present invention also provides a method of reducing free radical damage to sperm DNA in a male subject, the method including the steps of administering to the male subject:
(i) an effective amount of an anti-oxidant agent; and
(ii) an effective amount of an agent that reduces inflammation in the male reproductive tract and/or an effective amount of an agent that increases testicular testosterone concentration.

The present invention also provides a method of improving sperm production in a male subject, the method including the steps of administering to the male subject:
(i) an effective amount of an anti-oxidant agent; and
(ii) an effective amount of an agent that reduces inflammation in the male reproductive tract and/or an effective amount of an agent that increases testicular testosterone concentration.

The present invention also provides a method of improving fertility in a male subject, the method including the steps of administering to the male subject:
(i) an effective amount of an anti-oxidant agent; and
(ii) an effective amount of an agent that reduces inflammation in the male reproductive tract and/or an effective amount of an agent that increases testicular testosterone concentration.

The present invention also provides a method of treating infertility in a male subject, the method including the steps of administering to the male subject:
(i) an effective amount of an anti-oxidant agent; and
(ii) an effective amount of an agent that reduces inflammation in the male reproductive tract and/or an effective amount of an agent that increases testicular testosterone concentration.

The present invention also provides a method of improving quality of an embryo produced by fertilization of an oocyte by a sperm from a male subject, the method including the steps of administering to the male subject prior to fertilization of the oocyte with the sperm from the male subject:
(i) an effective amount of an anti-oxidant agent; and
(ii) an effective amount of an agent that reduces inflammation in the male reproductive tract and/or an effective amount of an agent that increases testicular testosterone concentration.

The present invention also provides a method of improving development of an embryo produced by fertilization of an oocyte by a sperm from a male subject, the method including the steps of administering to the male subject prior to fertilization of the oocyte with the sperm from the male subject:
(i) an effective amount of an anti-oxidant agent; and
(ii) an effective amount of an agent that reduces inflammation in the male reproductive tract and/or an effective amount of an agent that increases testicular testosterone concentration.

The present invention also provides a method of reducing the extent of DNA damage in a subject inherited from the father of the subject, the DNA damage being due to free radical damage to sperm DNA in the father of the subject, the method including the steps of administering to the father prior to conception of the subject:
(i) an effective amount of an anti-oxidant agent; and
(ii) an effective amount of an agent that reduces inflammation in the male reproductive tract and/or an effective amount of an agent that increases testicular testosterone concentration.

The present invention also provides a method of preventing a disease or condition in a subject, the disease or condition associated with DNA damage inherited from the father of the subject due to free radical damage to sperm DNA, the method including the steps of administering to the father prior to conception of the subject:
(i) an effective amount of an anti-oxidant agent; and
(ii) an effective amount of an agent that reduces inflammation in the male reproductive tract and/or an effective amount of an agent that increases testicular testosterone concentration.

The present invention also provides a method of increasing testosterone concentration in a male subject, the method including the steps of administering to the male subject:
(i) an effective amount of an anti-oxidant agent; and
(ii) an effective amount of an agent that reduces inflammation in the male reproductive tract and/or an effective amount of an agent that increases testicular testosterone concentration.

The present invention also provides a composition including the following components:
Vitamin E;
Vitamin C, or a salt thereof;
Lycopene;
Selenium;
Zinc; and
greater than 500 mg Garlic, or an extract or oil thereof, or a pharmaceutically acceptable derivative of any of the aforementioned components; the composition optionally further including folic acid, or a salt thereof, and/or Co Enzyme Q10.

The present invention also provides a composition including:
about 400 I.U. Vitamin E;
about 100 mg Vitamin C, or a salt thereof;
about 6 mg Lycopene;
about 26 μg Selenium;
about 25 mg Zinc;
about 40 mg Co-Enzyme Q10; and
about 1000 mg Garlic.

The present invention also provides a composition including:
about 400 I.U. Vitamin E;
about 100 mg Vitamin C, or a salt thereof;
about 6 mg Lycopene;
about 26 μg Selenium;
about 25 mg Zinc;
about 500 μg Folate; and
about 1000 mg Garlic.

The present invention also provides a combination product including the following components:
an anti-oxidant agent; and
an agent that reduces inflammation in the male reproductive tract; and/or
an agent that increases testicular testosterone concentration;
   wherein the said components in the combination product are not the same, and the components are provided in a form for separate administration to the subject, or in a form for co-administration of one or more of the components to the subject.

The present invention also provides a composition including:
(i) an effective amount of an anti-oxidant agent; and
(ii) an effective amount of an agent that reduces inflammation in the male reproductive tract and/or an effective amount of an agent that increases testicular testosterone concentration.

The present invention also provides a method of isolating sperm from a male subject, the method including the steps of:
(i) administering to the male subject an effective amount of an anti-oxidant agent;
(ii) administering to the male subject an effective amount of an agent that reduces inflammation in the male reproductive tract and/or an effective amount of an agent that increases testicular testosterone concentration; and
(iii) isolating sperm from the male subject.

The present invention arises from the finding that a composition including at least one anti-oxidant, in conjunction with an agent that reduces inflammation in the male reproductive tract (for example an agent that reduces inflammation in the male reproductive tract) and/or an agent that increases testicular testosterone concentration, when administered to a male subject is effective at improving the rate of natural and assisted pregnancy in a female subject fertilized with sperm from the male subject.

Without being bound by theory, it appears that the improvement in pregnancy rate is due to the fact that administration of at least one anti-oxidant, in conjunction with administration of an agent that reduces inflammation in the male reproductive tract and/or an agent that increases testicular testosterone concentration, is effective at reducing free radical damage to sperm in the male subject, thereby increasing the pregnancy rate.

Various terms that will be used throughout the specification have meanings that will be well understood by a skilled addressee. However, for ease of reference, some of these terms will now be defined.

The term “anti-oxidant agent” as used throughout the specification is to be understood to mean a molecule that can directly or indirectly reduce the damaging effects of oxygen and/or free-radicals in cells, and includes molecules that react with oxygen, or molecules that may protect against, and/or react with, a free radical.

In this regard, it will be understood that the term “anti-oxidant agent” includes a pro-drug, or an agent that when administered to a subject forms, or is metabolized to, an agent that has anti-oxidant activity. It will also be understood that the present invention includes for each of the exemplified anti-oxidant agents, a salt of the anti-oxidant (if applicable), or a pharmaceutically acceptable chemical derivative of the anti-oxidant agent.

The term “an agent that reduces inflammation in the male reproductive tract” as used throughout the specification is to be understood to mean a molecule that directly or indirectly results in a reduction in inflammation in the male reproductive tract. Such as agent may for example, directly or indirectly decrease leukocyte numbers, leukocyte proliferation or leukocyte activity in the male reproductive tract, and/or directly indirectly result in a reduction in the production of one or more inflammatory cytokines, such as TNF-α and IL-1β, by leukocytes when administered to a subject. The term includes a pro-drug or an agent that when administered to a subject forms, or is metabolized to, an agent that reduces inflammation in the male reproductive tract. It will also be understood that the present invention includes for each of the exemplified agents, a salt of the agent (if applicable), or a pharmaceutically acceptable chemical derivative of the agent.

In this regard, the male reproductive tract will be understood to include the epididymis, the penis, the prostate gland, the seminal vesicles, the testes, the vas deferens and semen.

The term “an agent that increases testicular testosterone concentration” as used throughout the specification is to be understood to mean a molecule that directly or indirectly results in an increase in testosterone concentration in the testes. The term includes a pro-drug or an agent that when administered to a subject forms, or is metabolized to, an agent that increases testicular testosterone concentration. It will also be understood that the present invention includes for each of the exemplified agents, a salt of the agent (if applicable), or a pharmaceutically acceptable chemical derivative of the agent.

### BRIEF DESCRIPTION OF THE FIGURES

FIG. 1 shows the effect of the OSMI nutraceutical on total motile sperm count in a group of men with known free radical damage.

FIG. 2 shows the effect of the OSMI nutraceutical on sperm membrane integrity using the HOST assay in a group of men with known free radical damage.

FIG. 3 shows the effect of the OSMI nutraceutical on sperm DNA fragmentation using TUNEL in a group of men with known free radical damage.

FIG. 4 shows the effect of the OSMI nutraceutical on sperm membrane lipid peroxidation using the TBARS assay.

### GENERAL DESCRIPTION OF THE INVENTION

As described above, in one embodiment the present invention provides a method of increasing pregnancy rate in a female subject, the female subject or an oocyte for introduction into the female subject fertilized by a sperm from a male subject, the method including the steps of administering to the male subject prior to fertilization:
(i) an effective amount of an anti-oxidant agent; and
(ii) an effective amount of an agent that reduces inflammation in the male reproductive tract and/or an effective amount of an agent that increases testicular testosterone concentration.

The present invention is based on the finding that administration of at least one anti-oxidant agent to a male subject, in conjunction with administration of an agent that reduces inflammation in the male reproductive tract and/or an agent that increases testicular testosterone concentration, is effective at improving the rate and outcome of natural and assisted pregnancies in females fertilized with sperm from the male subject.

The present invention may therefore be used to improve the rate and outcome of natural pregnancies (a naturally conceived pregnancy) and assisted pregnancies (a pregnancy produced by an assisted reproduction technology).

Examples of assisted reproduction technologies include artificial insemination, in vitro fertilization, gamete intrafallopian transfer (GIFT), intra-uterine insemination (IUI), intracytoplasmic sperm injection (ICSI), testicular sperm extraction (TESE), and percutanenous epididymal sperm aspiration (PESA). Methods for using assisted reproduction technologies in humans and animals are known in the art, including methods for isolating oocytes to be fertilized.

Methods for determining pregnancy rate are known in the art. Generally, pregnancy rate is a measure of the likelihood that a particular female subject will achieve an identifiable pregnancy, by either natural or assisted means. An identifiable pregnancy is a successful pregnancy as measured by one or more specific outcomes, such as positive βHCCG, or the detection of a viable fetal heart on first trimester scan (generally referred to as the viable pregnancy rate).

In the context of an individual subject receiving treatment according to the present invention, the pregnancy rate is therefore the likelihood that the subject is likely to achieve an identifiable pregnancy after fertilization. An increase in the pregnancy rate signifies an improved likelihood that the subject will achieve an identifiable pregnancy as compared to the situation where the subject is not receiving treatment.

In the context of a population of subjects, the pregnancy rate is the proportion of female subjects that achieve an identifiable pregnancy while undergoing the treatment of the present invention. For example, in the case of IVF pregnancies, the pregnancy rate may be measured as the proportion of viable pregnancies obtained upon transfer of an embryo.

In addition, the outcome for natural and assisted pregnancy is improved for couples in which the male partner has been prior treated with at least one anti-oxidant agent, in conjunction with administration of an agent that reduces inflammation in the male reproductive tract and/or an agent that increases testicular testosterone concentration.

Accordingly, in another embodiment the present invention provides a method of improving pregnancy outcome in a female subject, the female subject or an oocyte for introduction into the female subject fertilized by a sperm from a male subject, the method including the steps of administering to the male subject prior to fertilization:
(i) an effective amount of an anti-oxidant agent; and
(ii) an effective amount of an agent that reduces inflammation in the male reproductive tract and/or an effective amount of an agent that increases testicular testosterone concentration.

Methods for determining pregnancy outcome are known in the art. Generally, pregnancy outcome is an identifiable result associated with a natural or assisted pregnancy, such as a successful pregnancy as measured by one or more specific parameters (eg positive βHCCG, or the detection of a viable fetal heart on first trimester scan), the likelihood of the pregnancy being taken to term a viable birth, or the likelihood of the subject not suffering a miscarriage.

The administration of an anti-oxidant agent to a male subject, in conjunction with administration of an agent that reduces inflammation in the male reproductive tract and/or an agent that increases testicular testosterone concentration, may also reduce free radical damage to sperm from the male subject.

Accordingly, in another embodiment the present invention provides a method of reducing free radical damage to sperm produced by a male subject, the method including the steps of administering to the male subject:
(i) an effective amount of an anti-oxidant agent; and
(ii) an effective amount of an agent that reduces inflammation in the male reproductive tract and/or an effective amount of an agent that increases testicular testosterone concentration.

The administration of the anti-oxidant agent, in conjunction with an agent that reduces inflammation in the male reproductive tract and/or an agent that increases testicular testosterone concentration, may also reduce free radical levels in the male reproductive tract and in semen.

Accordingly, in another embodiment the present invention provides a method of reducing generation of free radicals in the reproductive tract and/or in semen of a male subject, the method including the steps of administering to the male subject:
(i) an effective amount of an anti-oxidant agent; and
(ii) an effective amount of an agent that reduces inflammation in the male reproductive tract and/or an effective amount of an agent that increases testicular testosterone concentration.

Without being bound by theory, it appears that administration of an anti-oxidant agent, in conjunction with administration of an agent that reduces inflammation in the male reproductive tract and/or an agent that increases testicular testosterone concentration, is effective in reducing free radical damage by one or more of the following mechanisms: (i) directly reducing the levels of free-radicals in the male reproductive tract and/or in semen; (ii) reducing the levels of free radicals produced in the male reproductive tract and/or in semen by leukocytes, by reducing leukocyte inflammatory cytokine production; and (iii) augmenting testosterone concentration in the testes by reducing free radical damage to the testosterone producing Leydig cells, thereby increasing testosterone levels and improving sperm function.

Administration of an anti-oxidant agent, in conjunction with an agent that reduces inflammation in the male reproductive tract and/or an agent that increases testicular testosterone concentration, may also result in improvement in the sperm count of the male subjects, improvement in sperm motility, improvement in sperm membrane integrity, and a reduction in DNA damage in the sperm.

It is also contemplated that the quality of embryos, as measured for example by the ability of embryos to form blastocyts, may also be improved using sperm from male subjects that have been prior treated with an anti-oxidant agent, in conjunction with an agent that reduces inflammation in the male reproductive tract and/or an agent that increases testicular testosterone concentration.

In this regard, the present invention may be used to improve the quality of embryos resulting from a natural conception or resulting from an assisted reproduction technology.

High levels of free radicals are generally present in semen of men with infertility of unknown origin, smokers, infertility related to poor sperm motility and in men following vasectomy reversal.

Without being bound by theory, it appears that free radicals are directly capable of interfering with male fertility by at least three mechanisms. Firstly, free radical damage to the sperm membrane interferes with function of the sperm tail, reducing sperm motility. Secondly, free radical damage to the headpiece can interfere with the acrosome reaction, the natural response vital to oocyte-sperm fusion and fertilization. Finally, if semen free radical levels are sufficiently high they can damage the sperm genetic material, leading to poor embryo quality and infertility/miscarriage.

In addition, sperm DNA damage has been linked to the development of childhood cancers in their progeny. Several studies, for example Ji et al. (1997) J. Natl. Cancer Inst. 89(3):238-244 and Sun et al. (1997) Biol. Reprod. 56:602-607, indicate that sperm DNA damage caused by the father smoking is linked to the development of childhood cancers in their progeny. Thus, prior treatment of a male subject in accordance with the present invention may lead to a decrease in DNA damage in the progeny of the male subject, and consequently a reduction in diseases and/or conditions in the progeny associated with inherited DNA damage.

The male subject in the various embodiments of the present invention may be for example a male human, or a male mammal including a primate, a livestock animal (eg. a horse, a cow, a sheep, a pig, a goat), a companion animal (eg. a dog, a cat), a laboratory test animal (eg. a mouse, a rat, a guinea pig), or any other male animal in which free radicals are generated by leukocytes in the reproductive tract and/or semen. In one embodiment, the male subject is a male human.

Accordingly, it will be appreciated that the present invention extends to the use in both humans and animals, and as such the present invention may be used in either humans or animals for natural conception purposes and for assisted reproduction purposes.

In this regard, the present invention is suitable for assisted reproduction technologies such as artificial insemination, in vitro fertilization (IVF; extraction of an oocyte, fertilization in the laboratory and transfer of the embryo into a recipient), gamete intrafallopian transfer (GIFT; placement of oocytes and sperm into the fallopian tube), intra-uterine insemination (IUI), intracytoplasmic sperm injection (ICSI), testicular sperm extraction (TESE), and percutanenous epididymal sperm aspiration (PESA).

The present invention also provides sperm (and/or semen) isolated from a male subject treated according to the present invention. Such sperm (eg as isolated sperm or in the form of a semen sample) may be used in both humans and animals for assisted reproduction. Methods for isolating sperm from humans and animals are known in the art.

Accordingly, in another embodiment the present invention provides a method of isolating sperm from a male subject, the method including the steps of:
(i) administering to the male subject an effective amount of an anti-oxidant agent;
(ii) administering to the male subject an effective amount of an agent that reduces inflammation in the male reproductive tract and/or an effective amount of an agent that increases testicular testosterone concentration; and
(iii) isolating sperm from the male subject.

The present invention also provides sperm (or semen) isolated from the male subject, and a non-human animal arising from fertilization of a female non-human animal, or a non-human animal arising from fertilization of an oocyte, with the sperm. Methods for isolating sperm are known in the art. Methods for producing non-human animals by fertilization are known in the art.

The sperm so isolated are also likely to better resist the effects of freezing and thawing. In this regard, cryopreservation and thawing are associated with a significant reduction in sperm function, including sperm motility, induced by oxidative stress. Thus, the present invention also extends to a method of improving the cryopreservation of sperm by treating a male subject with an effective amount of an anti-oxidant agent and an effective amount of an agent that reduces inflammation in the male reproductive tract and/or an effective amount of an agent that increases testicular testosterone concentration.

In one embodiment, the male subject of the present invention is selected from the group consisting of a subject with increased levels of sperm membrane oxidation, including a subject with increased levels of malondialdehyde or other biochemical markers of oxidative stress; a smoker; a subject with reduced fertility, including reduced fertility due to poor sperm motility, or reduced fertility of unknown origin; a subject having undergone vasectomy reversal; a subject with a reproductive tract infection such as epididymitis; and a subject having a varicocele.

In one embodiment, the free radical damage occurring to the sperm of the present invention is damage mediated by free radicals generated and/or present in the male reproductive systems, including in semen. In a further embodiment, the free radical damage is damage is due to free radicals generated by leukocytes and/or sperm in the male reproductive tract and/or in semen.

Methods are known in the art for assessing the extent of free radical damage to sperm. For example, the TBARs assay (which involves the measurement of malondialdehyde, a marker of sperm membrane oxidation) or LPO-856 spectrophotometric assay may be used. These methods are described for example in Gomez et al (1998) International Journal of Andrology 21(2):81-96 and Aitken et al. (1993) Molecular Reproduction and Development 35:302-315.

In one embodiment, the administration to the subject results in a reduction in free radical damage to sperm of 10% or greater of the level of malondialdehyde (pmol per 10⁷ sperm) measured in the sperm as compared to before administration. In another embodiment, the administration to the subject results in a reduction in free radical damage to sperm of 15% or greater of the level of malondialdehyde (pmol per 107 sperm) measured in the sperm as compared to before administration.

The anti-oxidant agent in the various embodiments of the present invention may be one or more individual anti-oxidants. In this regard, an anti-oxidant is a molecule that can directly or indirectly reduce the damaging effects of oxygen and/or free-radicals in cells, and includes molecules that react with oxygen, or molecules that may protect against, and/or react with, a free radical.

In one embodiment, the anti-oxidant agent is selected from one or more of the group consisting of a β-carotenoid, including lycopene (a carotenoid derived from the skin of tomato), lutein, and zeaxanthin; Vitamin C; Vitamin E; Co-Enzyme Q10; selenium; zinc; L-carnitine; acetylcarnitene; N-acetylcysteine; glutathionine; pyruvate; and hypotaurine; or a salt (if applicable), or a pharmaceutically acceptable derivative of any of the aforementioned agents.

Other anti-oxidants are generally as described Agarwal et al (2004) Reproductive Biomedicine Online 8(6): 616-27.

The effective amount of the one or more anti-oxidant agents in the various embodiments of the present invention is not particularly limited, so long as it has the desired or therapeutic effect, and will depend upon the particular anti-oxidant(s) administered.

In this regard, suitable concentrations for a number of the anti-oxidant agents in the various embodiments of the present invention are as follows:

Vitamin E (d-alpha-tocopheryl acetate): 40 to 4000 I.U, with a usual range of 200 to 1200 I.U., and typically 200 to 600 I.U.
Vitamin C (ascorbic acid or a salt thereof): 10 to 1000 mg, with a usual range of 20 to 200 mg.
Lycopene: 0.5 to 50 mg, with usual ranges of 1 to 20 mg, and 2 to 10 mg.
Co-Enzyme Q10: 4 to 400 mg, with a usual range of 10 to 100 mg.
Selenium: 10 to 250 μg, with a usual range of 20 to 50 μg.
Zinc: 2.5 to 100 mg, with a usual range of 10 to 50 mg.
Glutathione: 100 to 1000 mg, with a usual range of 400 to 600 mg.
L-carnitene: 1 to 5 grams, with a usual range of 2 to 3 grams.
Pentoxifylline: 200 to 1500 mg, with a usual range of 300 to 1200 mg.

In one embodiment, the anti-oxidant agent administered to the subject is a combination of the following anti-oxidant agents: lycopene, Vitamin C, Vitamin E, selenium and zinc. The anti-oxidant Co-Enzyme Q10 may also be administered.

In one embodiment, the agent that reduces inflammation in the male reproductive tract is an agent that reduces leukocyte number, proliferation and/or production, and/or an agent that inhibits leukocyte cytokine production.

Examples of agents that reduce inflammation in the male reproductive tract, such as agents that inhibit leukocyte cytokine production, include:

Garlic (as described in Hodge et al (2002) Cytometry 48: 209-215), or an oil, extract or active compound derived therefrom.
Green tea or an extract or active compound derived therefrom.
N-3 fatty acids (as described in Weiss et al (2002) British Journal of Nutrition 87(sup 1): S89-94).
Docosahexaenoic acid (as described in Kelley et al. (1999) Lipids 34(4):317-24), or a salt or pharmaceutically acceptable derivative thereof.
Ginger and its derivatives (eg Zerumbone) (as described in Murakami et al (2002) Carcinogenesis 23(5):795-802), or an extract or active compound derived therefrom.
Agents that decrease leukocyte production of TNFα, such as pentoxyphylline (as described in Meiners et al (2004) J Neural Transm 111(3): 441-447).
Agents that block the action of TNFα once produced, such as infliximab.
Lipid extract from marine mollusk (Lyprinol-Blackmores), a 5-Lipoxygenase inhibitor (as described in Dugas B (2000). Allerg Immunol (Paris) 32(7):284-9).

In one embodiment, the agent that reduces inflammation in the male reproductive tract also inhibits leukocyte proliferation. Examples of such agents are garlic (or an oil, extract or active compound derived therefrom), or ginger (or an extract, derivative or active compound derived therefrom).

Examples of agents that increase testicular testosterone concentration in the various embodiments of the present invention include:

Garlic (as described in Oi et al (2001) J. Nutr. 131(8):2150-2156), or an oil, extract or active compound derived therefrom.
Zinc (as described in Fuse et al (1999) Int. Urol. Nephrol. 31(3):401-408.
acetylcamitine.
tribulus terrestris, or an extract or active compound derived therefrom.
Agents that reduce inducible nitric oxide synthase (iNOS) in macrophages, such as allicin or ajoene, active compounds derived from garlic.
Agents that reduce nitric oxide (NO) production.

In one embodiment, the agent that increases testosterone concentration of the present invention also inhibits leukocyte proliferation.

A suitable method of determining whether an agent increases testicular testosterone concentration is by way of determining the concentration of testosterone in seminal plasma, for example, as described in Luboshitzky et al (2002) Int. J. Androl. 25(6):345.

In one embodiment, the agent that reduces inflammation in the male reproductive tract and the agent that increases testicular testosterone concentration of the present invention are the same agent with both of these activities. Examples of such agents are garlic and zinc.

The effective amount of the agent that reduces inflammation in the male reproductive tract and/or the effective amount of the agent that increases testicular testosterone concentration is not particularly limited, so long as it has the desired or therapeutic effects, and will depend upon the particular agents administered.

In one embodiment, the agent that reduces inflammation in the male reproductive tract and/or the agent that increases testicular testosterone concentration is garlic, garlic oil, a garlic extract, or an active component derived from garlic, such as allicin and/or ajoene. A suitable garlic extract may be produced by taking fresh garlic, shelling and crushing the garlic, and filtering the crushed extract through a series of filters.

In the case of garlic oil, an effective amount for administration in combination with the anti-oxidant is greater than 500 mg, and typically 501 to 10,000 mg. In one embodiment, the amount of garlic oil administered is 1000 mg or about this amount.

The anti-oxidant agent, and the agent that reduces inflammation in the male reproductive tract and/or the agent that increases testicular testosterone concentration in the various embodiments of the present invention, may be administered to the subject separately or in combination, in accordance with a suitable administration regime.

Thus, the administration may be sequential or simultaneous and generally means that the pharmaceutical compositions are present in the subject during a specified time interval. Typically, if an agent is administered within the half-life of the first agent, the agents are considered co-administered.

Accordingly, in another embodiment the present invention provides a combination product including the following components:
an anti-oxidant agent; and
an agent that reduces inflammation in the male reproductive tract; and/or
an agent that increases testicular testosterone concentration;
   wherein the said components in the combination product are not the same, and the components are provided in a form for separate administration to the male subject, or in a form for co-administration of one or more of the components to the male subject.

The combination product may be used for the various applications of the present invention as described herein.

In one embodiment, the combination product is used to improve pregnancy rate, to improve pregnancy outcome, or to reduce free radical damage to sperm produced by a male subject, as discussed herein.

The anti-oxidant agent, and the agent that reduces inflammation in the male reproductive tract and/or the agent that increases testicular testosterone concentration, in the various combination products of the present invention may be packaged separately in suitably sterilized containers such as ampoules, bottles, or vials, either in multi-dose or in unit dosage forms. The containers are generally hermetically sealed after being filled. Alternatively, the various components may be packaged for co-administration of one or more of the components together. Methods for packaging the various components are known in the art.

In one embodiment, the anti-oxidant agent and the agent that reduces inflammation in the male reproductive tract and/or the agent that increases testicular testosterone concentration are administered to the subject as a composition in the form of a single formulation.

Accordingly, in another embodiment the present invention provides a composition including:
(i) an effective amount of an anti-oxidant agent; and
(ii) an effective amount of an agent that reduces inflammation in the male reproductive tract and/or an effective amount of an agent that increases testicular testosterone concentration.

The composition may be used for the various applications of the present invention as described herein.

In one embodiment, the composition is used for administration to a male subject to improve pregnancy rate, to improve pregnancy outcome, or to reduce free radical damage to sperm produced by a male subject, as discussed herein.

As discussed previously herein, in one embodiment the agent that reduces inflammation in the male reproductive tract also inhibits leukocyte proliferation.

In another embodiment, the present invention provides the use of an anti-oxidant agent, in combination with an agent that reduces inflammation in the male reproductive tract and/or an agent that increases testicular testosterone concentration, in the preparation of a medicament for the various applications of the present invention described herein, such as for improving pregnancy rate, for improving pregnancy outcome, to reduce free radical damage to sperm produced by a male subject.

A suitable composition (referred to as the OSMI formulation) is as follows:

Vitamin E (d-alpha-tocopheryl acetate), 400 I.U.;
Vitamin C (ascorbic acid or a salt thereof) 100 mg;

### Lycopene 6 mg;

### Co-Enzyme Q10 40 mg;

### Selenium 26 μg;

### Zinc 25 mg; and

Garlic Oil 1000 mg, or a composition with about the above amounts of the various components.

The above formulation (nutraceutical) may be administered, for example, in the form of a capsule for oral delivery to the subject.

The administration to the subject of the composition or combination product of the various embodiments of the present invention may also include separate administration or co-administration of other agents. For example, agents that enhance sperm function and/or agents that are involved in cellular DNA synthesis may also be administered to the subject to reduce the extent of free radical damage to sperm in a male subject. An example of an agent that improves sperm function and plays an important role in cellular DNA synthesis is folate. An example of an agent that boosts sperm function is L-carnitene.

A suitable formulation (referred to as the Menevit formulation) including folate is as follows:

Vitamin E (d-alpha-tocopheryl acetate), 400 I.U.
Vitamin C (ascorbic acid or a salt thereof) 100 mg

### Lycopene 6 mg

### Folate 500 μg

### Selenium 26 μg

### Zinc 25 mg

Garlic Oil 1000 mg, or a composition with about the above amounts of the various components.

The above formulation (nutraceutical) may be administered, for example, in the form of a capsule for oral delivery to the subject. The above formulation may also include 40 mg of Co-Enzyme Q10.

Accordingly, in another embodiment the present invention provides a composition including:
Vitamin E;
Vitamin C, or a salt thereof;
Lycopene;
Selenium;
Zinc; and
greater than 500 mg Garlic, or an extract or oil thereof; the composition optionally further including folic acid, or a salt thereof, and/or Co Enzyme Q10; or a pharmaceutically acceptable derivative of any of the aforementioned.

The administration to the subject of the anti-oxidant agent, and administration of the agent that reduces inflammation in the male reproductive tract and/or the agent that increases testicular testosterone concentration, in the various embodiments of the present invention will be in a suitable form to the effect the desired outcome, such as an improvement in pregnancy rate or outcome, or a reduction in free radical damage to sperm. The effective amount of each of the anti-oxidant agent and the other agents to be administered is not particularly limited, so long as it is within such an amount and in such a form that generally exhibits the desired or therapeutic effect.

In this regard, an effective amount of the anti-oxidant and the other agent(s) may be appropriately chosen, depending upon, for example, the type and extent of reduced fertility to be treated, the age and body weight of the subject, the frequency of administration, and the presence of other active agents.

In one embodiment, the frequency of administration of the anti-oxidant, and the agent that reduces inflammation in the male reproductive tract and/or the agent that increases testicular testosterone concentration, is a daily administration.

In one embodiment, the duration of the administration regime is for 6 weeks or longer. In another embodiment, the duration of the administration regime is for 10 weeks or longer. In a further embodiment, the duration of the administration regime is for 12 weeks or longer.

In this regard, a suitable administration regime for either of the following formulations is one administration per day of one of the following formulations for a period of 12 weeks:

### OSMI Formulation:

Vitamin E (d-alpha-tocopheryl acetate), 400 I.U.
Vitamin C (ascorbic acid or a salt thereof) 100 mg

### Lycopene 6 mg

### Co-Enzyme Q10 40 mg

### Selenium 26 μg

### Zinc 25 mg

### Garlic Oil 1000 mg

### Menevit Formulation:

Vitamin E (d-alpha-tocopheryl acetate), 400 I.U.
Vitamin C (ascorbic acid or a salt thereof) 100 mg

### Lycopene 6 mg

### Folate 500 μg

### Selenium 26 μg

### Zinc 25 mg

### Garlic Oil 1000 mg

However, it will be appreciated that the administration of the anti-oxidant agent and the other agents in the various embodiments of the present invention may be within any time and frequency suitable to produce the desired effect. The anti-oxidant and the other agents may be administered orally, parenterally, topically or by any other suitable means.

Formulation of the agents of the present invention and their administration may be achieved by a suitable method known in the art.

For example, the administration of the anti-oxidant agent and the other agents in the various embodiments of the present invention may also include the use of one or more pharmaceutically acceptable additives, including pharmaceutically acceptable salts, amino acids, polypeptides, polymers, solvents, buffers, excipients and bulking agents, taking into consideration the particular physical and chemical characteristics of the various agents to be administered.

For example, the anti-oxidant agent and the other agents may be separately or jointly be prepared into a variety of pharmaceutical compositions in the form of, e.g., an aqueous solution, an oily preparation, a fatty emulsion, an emulsion, a gel, etc., and these preparations can be administered as intramuscular or subcutaneous injection or as injection to an organ (including the heart), or as an embedded preparation or as a transmucosal preparation through nasal cavity, rectum, lung, etc.

As discussed previously herein, the agents in the various embodiments of the present invention may be administered jointly or separately in the form of oral preparations (for example solid preparations such as tablets, capsules, granules or powders; liquid preparations such as syrup, emulsions or suspensions). Compositions containing the anti-oxidant agent and the other agents separately or jointly in the various embodiments of the present invention may also contain a preservative, stabiliser, dispersing agent, pH controller or isotonic agent. Examples of suitable preservatives in the various embodiments of the present invention are glycerin, propylene glycol, phenol or benzyl alcohol. Examples of suitable stabilisers in the various embodiments of the present invention are dextran, gelatin, a-tocopherol acetate or alpha-thioglycerin. Examples of suitable dispersing agents in the various embodiments of the present invention include polyoxyethylene (20), sorbitan mono-oleate (Tween 80), sorbitan sesquioleate (Span 30), polyoxyethylene (160) polyoxypropylene (30) glycol (Pluronic F68) or polyoxyethylene hydrogenated castor oil 60. Examples of suitable pH controllers in the various embodiments of the present invention include hydrochloric acid, sodium hydroxide and the like. Examples of suitable isotonic agents are glucose, D-sorbitol or D-mannitol.

The administration of the anti-oxidant agent and the other agents separately or jointly in the various embodiments of the present invention may also be in the form of a composition containing a pharmaceutically acceptable carrier, diluent, excipient, suspending agent, lubricating agent, adjuvant, vehicle, delivery system, emulsifier, disintegrant, absorbent, preservative, surfactant, colorant, flavorant or sweetener, taking into account the physical and chemical properties of the anti-oxidant agent and the other agents being administered.

For these purposes, the agents may be administered orally, parenterally, by inhalation spray, adsorption, absorption, topically, rectally, nasally, bucally, via an implanted reservoir in dosage formulations containing conventional non-toxic pharmaceutically-acceptable carriers, or by any other convenient dosage form. The term parenteral as used herein includes subcutaneous, intravenous, intramuscular, intraperitoneal, intrathecal, intrasternal, and intracranial injection or infusion techniques.

When administered parenterally, the administration of the anti-oxidant agent and the other agents separately or jointly will normally be in a unit dosage, sterile injectable form (solution, suspension or emulsion) that is typically isotonic with the blood of the recipient with a pharmaceutically acceptable carrier. Examples of such sterile injectable forms are sterile injectable aqueous or oleaginous suspensions. These suspensions may be formulated according to techniques known in the art using suitable dispersing or wetting agents and suspending agents. The sterile injectable forms may also be sterile injectable solutions or suspensions in non-toxic parenterally-acceptable diluents or solvents, for example, as solutions in 1,3-butanediol. Among the acceptable vehicles and solvents in the various embodiments of the present invention that may be employed are water, saline, Ringer's solution, dextrose solution, isotonic sodium chloride solution, and Hanks' solution. In addition, sterile, fixed oils are conventionally employed as solvents or suspending mediums. For this purpose, any bland fixed oil may be employed including synthetic mono- or di-glycerides, corn, cottonseed, peanut, and sesame oil. Fatty acids such as ethyl oleate, isopropyl myristate, and oleic acid and its glyceride derivatives, including olive oil and castor oil, especially in their polyoxyethylated versions, are useful in the preparation of injectables. These oil solutions or suspensions may also contain long-chain alcohol diluents or dispersants.

The carrier in the various embodiments of the present invention may contain minor amounts of additives, such as substances that enhance solubility, isotonicity, and chemical stability, for example buffers and preservatives.

When administered orally, the anti-oxidant agent and/or the other agents will usually be formulated into unit dosage forms such as tablets, cachets, powder, granules, beads, chewable lozenges, capsules, liquids, aqueous suspensions or solutions, or similar dosage forms, using conventional equipment and techniques known in the art. Such formulations typically include a solid, semisolid, or liquid carrier. Exemplary carriers include lactose, dextrose, sucrose, sorbitol, mannitol, starches, gum acacia, calcium phosphate, mineral oil, cocoa butter, oil of theobroma, alginates, tragacanth, gelatin, syrup, methyl cellulose, polyoxyethylene sorbitan monolaurate, methyl hydroxybenzoate, propyl hydroxybenzoate, talc, magnesium stearate, and the like.

A tablet may be made by compressing or molding the anti-oxidant agent and/or the other agents optionally with one or more accessory ingredients. Compressed tablets may be prepared by compressing, in a suitable machine, the active ingredient in a free-flowing form such as a powder or granules, optionally mixed with a binder, lubricant, inert diluent, surface active, or dispersing agent. Moulded tablets may be made by molding in a suitable machine, a mixture of the powdered active ingredient and a suitable carrier moistened with an inert liquid diluent.

The administration of the anti-oxidant agent and/or the other agents in the various embodiments of the present invention may also utilize controlled release technology. The anti-oxidant agent and/or the other agents may also be administered as a sustained-release pharmaceutical. To further increase the sustained release effect, the anti-oxidant agent and/or the other agents may be formulated with additional components such as vegetable oil (for example soybean oil, sesame oil, camellia oil, castor oil, peanut oil, rape seed oil); middle fatty acid triglycerides; fatty acid esters such as ethyl oleate; polysiloxane derivatives; alternatively, water-soluble high molecular weight compounds such as hyaluronic acid or salts thereof (weight average molecular weight: ca. 80,000 to 2,000,000), carboxymethylcellulose sodium (weight average molecular weight: ca. 20,000 to 400,000), hydroxypropylcellulose (viscosity in 2% aqueous solution: 3 to 4,000 cps), atherocollagen (weight average molecular weight: ca. 300,000), polyethylene glycol (weight average molecular weight: ca. 400 to 20,000), polyethylene oxide (weight average molecular weight: ca. 100,000 to 9,000,000), hydroxypropylmethylcellulose (viscosity in 1% aqueous solution: 4 to 100,000 cSt), methylcellulose (viscosity in 2% aqueous solution: 15 to 8,000 cSt), polyvinyl alcohol (viscosity: 2 to 100 cSt), polyvinylpyrrolidone (weight average molecular weight: 25,000 to 1,200,000).

Alternatively, the anti-oxidant agent and the other agents in the various embodiments of the present invention may be separately or jointly incorporated into a hydrophobic polymer matrix for controlled release over a period of days. The anti-oxidant agent and/or the other agents may then be molded into a solid implant, or externally applied patch, suitable for providing efficacious concentrations of either or both the anti-oxidant agent and the other agents over a prolonged period of time without the need for frequent re-dosing. Such controlled release films are well known to the art. Other examples of polymers commonly employed for this purpose that may be used include nondegradable ethylene-vinyl acetate copolymer a degradable lactic acid-glycolic acid copolymers, which may be used externally or internally. Certain hydrogels such as poly(hydroxyethylmethacrylate) or poly(vinylalcohol) also may be useful, but for shorter release cycles than the other polymer release systems, such as those mentioned above.

The carrier in the various embodiments of the present invention may also be a solid biodegradable polymer or mixture of biodegradable polymers with appropriate time release characteristics and release kinetics. The anti-oxidant agent and/or the other agents may then be molded into a solid implant suitable for providing efficacious concentrations of the anti-oxidant and/or the other agents over a prolonged period of time without the need for frequent re-dosing. The anti-oxidant and/or the other agents may be incorporated into the biodegradable polymer or polymer mixture in any suitable manner known to one of ordinary skill in the art and may form a homogeneous matrix with the biodegradable polymer, or may be encapsulated in some way within the polymer, or may be molded into a solid implant.

It should also be appreciated that other methods of delivery of the anti-oxidant and/or the other agents in the various embodiments of the present invention are contemplated. For example, if the anti-oxidant and/or the other agents are protein species, they may be separately or jointly delivered by way of a nucleic acid or vector that allows for expression in the appropriate target cell. For example, they may be delivered by way of a viral vector that causes expression in target cells in the male reproductive tract. Examples of enzymatic anti-oxidants include superoxide dismutase, catalase and glutathione peroxidase.

As discussed previously herein, the present invention is also suitable for reducing the generation of free radicals in the male reproductive tract and/or semen.

In this regard, the male reproductive tract will be understood to include the epididymis, the penis, the prostate gland, the seminal vesicles, the testes, the vas deferens and semen.

Methods for determining the level of free radicals in the male reproductive tract are known in the art. For example, free radical production by sperm or seminal leukocytes can be measured directly using chemiluminescence assays (as described in Kobayashi et al (2001) J Androl 22(4):568-74). Alternatively, assays that measure free radical related damage to sperm lipid membrane may be used. For example, the TBARs assay (which involves the measurement of malondialdehyde, a marker of sperm membrane oxidation) or LPO-586 spectrophotometric assay may be used. These methods are described in Gomez et al (1998) International Journal of Andrology 21(2):81-96 and Aitken et al (1993) Molecular Reproduction and Development 35:302-315.

In one embodiment, the free radicals are generated by leukocytes and/or sperm present in the male reproductive tract and/or semen.

Examples of suitable anti-oxidant agents are as previously described herein. Examples of agents that inhibit leukocyte cytokine production, and/or agents that increase testicular testosterone concentration are as previously described herein.

The effective amount of an anti-oxidant agent, and the agent that reduces inflammation in the male reproductive tract and/or the amount of an agent that increases testicular testosterone concentration, is not particularly limited, so long as it has the desired or therapeutic effect, and will depend upon the particular agents administered. Suitable concentrations for the agents are as described previously herein.

An effective amount of the anti-oxidant agent and the other agents may be appropriately chosen, depending upon, for example, the type and extent of reduced fertility to be treated, the age and body weight of the subject, the frequency of administration, and the presence of other active agents.

The anti-oxidant, and the agent that reduces inflammation in the male reproductive tract and/or the agent that increases testicular testosterone concentration, may be administered to the subject separately or in combination.

Accordingly, in another embodiment the present invention provides a combination product for reducing generation of free radicals in the reproductive tract and/or in semen of a male subject, the combination product including the following components:
an anti-oxidant agent; and
an agent that reduces inflammation in the male reproductive tract; and/or
an agent that increases testicular testosterone concentration;
   wherein the said components in the combination product are not the same, and the components are provided in a form for separate administration to the subject, or in a form for co-administration of one or more of the components to the subject.

In one embodiment, the anti-oxidant, and the agent reduces inflammation in the male reproductive tract and/or the agent that increases testicular testosterone concentration, are administered to the subject as a composition in the form of a single formulation.

Accordingly, in another embodiment the present invention provides a composition for reducing generation of free radicals in the reproductive tract and/or in semen of a male, the composition including:
(i) an effective amount of an anti-oxidant agent; and
(ii) an effective amount of an agent that reduces inflammation in the male reproductive tract and/or an effective amount of an agent that increases testicular testosterone concentration.

In another embodiment, the present invention provides the use of an anti-oxidant agent, in combination with an agent that reduces inflammation in the male reproductive tract and/or an agent that increases testicular testosterone concentration, in the preparation of a medicament for reducing generation of free radicals in the reproductive tract and/or in semen of a male subject.

Suitable compositions are as previously described herein, namely the OSMI and Menevit formulations.

The administration of the anti-oxidant, and the administration of the agent that reduces inflammation in the male reproductive tract and/or the agent that increases testicular testosterone concentration, are as previously described herein.

As described previously, the present invention is also suitable for reducing the activity and/or concentration of leukocytes in the reproductive tract of a male subject.

Accordingly, in another embodiment the present invention provides a method of reducing activity and/or concentration of leukocytes in the reproductive tract and/or semen of a male subject, the method including the steps of administering to the male subject:
(i) an effective amount of an anti-oxidant agent; and
(ii) an effective amount of an agent that reduces inflammation in the male reproductive tract and/or an effective amount of an agent that that increases testicular testosterone concentration.

Methods for determining the level of activity and/or concentration of leukocytes in the male reproductive tract are known in the art. For example, the use of peroxidase staining of semen cellular slides or monoclonal antibodies towards leukocyte surface antigens such as CD45 may be used (as described in Henkel et al. (2003) Andrologia 35(5): 309-314). Alternatively, seminal cytokines such as IL-6 can be measured which correlate with oxidative stress and leukocyte activity within semen (as described in Nallella et al. (2004) Urology 64(5):1010-3).

Examples of suitable anti-oxidant agents are as previously described herein. Examples of agents that reduce inflammation in the male reproductive tract, and agents that increase testicular testosterone concentration, are as previously described herein.

The effective amount of an anti-oxidant agent, and an agent that reduces inflammation in the male reproductive tract and/or an agent that increases testicular testosterone concentration, is not particularly limited, so long as it has the desired or therapeutic effect, and will depend upon the particular agents administered. Suitable concentrations for the agents are as described previously.

An effective amount of the anti-oxidant agent and the other agents may be appropriately chosen, depending upon, for example, the type and extent of reduced fertility to be treated, the age and body weight of the subject, the frequency of administration, and the presence of other active agents.

The anti-oxidant agent, and the agent that reduces inflammation in the male reproductive tract and/or the agent that increases testicular testosterone concentration, may be administered to the subject separately or in combination.

Accordingly, in another embodiment the present invention provides a combination product for reducing activity and/or concentration of leukocytes in the reproductive tract and/or semen of a male subject, the combination product including the following components:
an anti-oxidant agent; and
an agent that reduces inflammation in the male reproductive tract; and/or
an agent that increases testicular testosterone concentration;
   wherein the said components in the combination product are note the same, and the components are provided in a form for separate administration to the subject, or in a form for co-administration of one or more of the components to the male subject.

In one embodiment, the anti-oxidant and the agent that reduces inflammation in the male reproductive tract and/or the agent that increases testicular testosterone concentration are administered to the subject as a composition in the form of a single formulation.

Accordingly, in another embodiment the present invention provides a composition for reducing activity and/or concentration of leukocytes in the reproductive tract and/or semen of a male subject, the composition including:
(i) an effective amount of an anti-oxidant agent; and
(ii) an effective amount of an agent that reduces inflammation in the male reproductive tract and/or an effective amount of an agent that increases testicular testosterone concentration.

In another embodiment, the present invention provides the use of an anti-oxidant agent, in combination with an agent that reduces inflammation in the male reproductive tract and/or an agent that increases testicular testosterone concentration, in the preparation of a medicament for reducing activity and/or concentration of leukocytes in the reproductive tract of a male subject.

Suitable compositions are as previously described herein, namely the OSMI and Menevit formulations.

The administration of the anti-oxidant, and the administration of the agent that reduces inflammation in the male reproductive tract and/or the agent that increases testicular testosterone concentration, are as previously described herein.

The present invention is also suitable for reducing the level and/or production of an inflammatory mediators in the reproductive tract, such as reducing the level and/or production of inflammatory cytokines in the reproductive tract of a male subject.

Accordingly, in another embodiment the present invention provides a method of reducing the level and/or production of an inflammatory cytokine in the reproductive tract and/or semen of a male subject, the method including the steps of administering to the male subject:
(i) an effective amount of an anti-oxidant agent; and
(ii) an effective amount of an agent that reduces inflammation in the male reproductive tract and/or an effective amount of an agent that increases testicular testosterone concentration.

In one embodiment, the inflammatory cytokine is one or more of IL-1, IL-6, IL-8, TNF-α and Interferon-γ.

Methods for determining the level of an inflammatory cytokine in the male reproductive tract are known in the art, such ELISA assays for detection of pro-inflammatory cytokines such as IL-1, IL-6, IL-8, TNF-α and Interferon-γ, as described in Depuydt et al. (1996) J Andrology 17(6):699-707, Maegawa et al (2002). J Reprod Immunology 54: 33-42, and Nallella et al. (2004) Urology 64(5): 1010-3.

Examples of suitable anti-oxidant agents are as previously described herein. Examples of agents that reduce inflammation in the male reproductive tract leukocyte and agents that increase testicular testosterone concentration, are as previously described herein.

The effective amount of an anti-oxidant, and the agent that reduces inflammation in the male reproductive tract and/or the agent that increases testicular testosterone concentration, is not particularly limited, so long as it has the desired or therapeutic effect, and will depend upon the particular agents administered. Suitable concentrations for the anti-oxidant and the other agents are as described previously herein.

An effective amount of the anti-oxidant agent and the other agents may be appropriately chosen, depending upon, for example, the type and extent of reduced fertility to be treated, the age and body weight of the subject, the frequency of administration, and the presence of other active agents.

The anti-oxidant, and the agent that reduces inflammation in the male reproductive tract and/or the agent that increases testicular testosterone concentration, may be administered to the subject separately or in combination.

Accordingly, in another embodiment the present invention provides a combination product for reducing inflammatory cytokine production in the reproductive tract and/or semen of a male subject, the combination product including the following components:
an anti-oxidant; and
an agent that reduces inflammation in the male reproductive tract; and/or
an agent that increases testicular testosterone concentration;
   wherein the said components in the combination product are not the same, and the components are provided in a form for separate administration to the subject, or in a form for co-administration of one or more of the components to the subject.

In one embodiment the anti-oxidant agent, and the agent that reduces inflammation in the male reproductive tract and/or the agent that increases testicular testosterone concentration, are administered to the subject as a composition in the form of a single formulation.

Accordingly, in another embodiment the present invention provides a composition for reducing inflammatory cytokine production in the reproductive tract and/or semen of a male subject, the composition including:
(i) an effective amount of an anti-oxidant agent; and
(ii) an effective amount of an agent that reduces inflammation in the male reproductive tract and/or an effective amount of an agent that increases testicular testosterone concentration.

In another embodiment, the present invention also provides the use of an anti-oxidant agent, in combination with an agent that reduces inflammation in the male reproductive tract and/or an agent that increases testicular testosterone concentration, in the preparation of a medicament for reducing inflammatory cytokine production in the reproductive tract of a male subject.

Suitable compositions are as previously described herein, namely the OSMI and Menevit formulations.

The administration of the anti-oxidant, and the administration of the agent that reduces inflammation in the male reproductive tract and/or the agent that increases testicular testosterone concentration, are as previously described herein.

The present invention is also suitable for improving sperm function in a male subject.

Accordingly, in another embodiment the present invention provides a method of improving sperm function in a male subject, the method including the steps of administering to the male subject:
(i) an effective amount of an anti-oxidant agent; and
(ii) an effective amount of an agent that reduces inflammation in the male reproductive tract and/or an effective amount of an agent that increases testicular testosterone concentration.

In this regard, the term “sperm function” is any key component of sperm physiology and includes swimming activity towards the oocyte (motility), ability to undergo capacitation to penetrate the oocyte's outer coat (zona pellucida) and fuse with the oocyte membrane, and maintenance of sperm DNA integrity to form a functional male pro-nucleus at syngamy.

Methods for determining the level of sperm are known in the art. For example, suitable methods are described in detail within the World Health Organisation (WHO) laboratory manual for the examination of human semen and sperm-cervical mucous interaction. 4th edition. Cambridge University Press 1999.

Examples of suitable anti-oxidant agents are as previously described herein. Examples of agents that reduce inflammation in the male reproductive tract and agents that increase testicular testosterone concentration, are as previously described herein.

The effective amount of an anti-oxidant, and the agent that reduces inflammation in the male reproductive tract and/or the agent that increases testicular testosterone concentration, is not particularly limited, so long as it has the desired or therapeutic effect, and will depend upon the particular agents administered. Suitable concentrations for the anti-oxidant agents and the other agents are as described previously herein.

An effective amount of the anti-oxidant agent and the other agents may be appropriately chosen, depending upon, for example, the type and extent of reduced fertility to be treated, the age and body weight of the subject, the frequency of administration, and the presence of other active agents.

The anti-oxidant agent, and the agent that reduces inflammation in the male reproductive tract and/or the agent that increases testicular testosterone concentration, may be administered to the subject separately or in combination.

Accordingly, in another embodiment the present invention provides a combination product for improving sperm function in a male subject, the combination product including the following components:
an anti-oxidant; and
an agent that reduces inflammation in the male reproductive tract; and/or
an agent that increases testicular testosterone concentration;
   wherein the said components in the combination product are not the same, and the components are provided in a form for separate administration to the subject, or in a form for co-administration of one or more of the components to the subject.

In one embodiment the anti-oxidant agent, and the agent that reduces inflammation in the male reproductive tract and/or the agent that increases testicular testosterone concentration, are administered to the subject as a composition in the form of a single formulation.

Accordingly, in another embodiment the present invention provides a composition for improving sperm function in a male subject, the composition including:
(i) an effective amount of an anti-oxidant agent; and
(ii) an effective amount of an agent that reduces inflammation in the male reproductive tract and/or an effective amount of an agent that increases testicular testosterone concentration.

In another embodiment, the present invention provides the use of an anti-oxidant agent, in combination with an agent that reduces inflammation in the male reproductive tract and/or an agent that increases testicular testosterone concentration, in the preparation of a medicament for improving sperm function in a male subject.

Suitable compositions are as previously described herein, namely the OSMI and Menevit formulations.

The administration of the anti-oxidant agent, and the administration of the agent that reduces inflammation in the male reproductive tract and/or the agent that increases testicular testosterone concentration, are as previously described herein.

The present invention is also suitable for improving sperm motility in a male subject.

Accordingly, in another embodiment the present invention provides a method of improving sperm motility in a male subject, the method including the steps of administering to the male subject:
(i) an effective amount of an anti-oxidant agent; and
(ii) an effective amount of an agent that reduces inflammation in the male reproductive tract and/or an effective amount of an agent that increases testicular testosterone concentration.

Methods for determining sperm motility are known in the art. For example, suitable methods are described in detail within the World Health Organisation (WHO) laboratory manual for the examination of human semen and sperm-cervical mucous interaction. 4th edition. Cambridge University Press 1999.

Examples of suitable anti-oxidant agents are as previously described. Examples of agents that reduce inflammation in the male reproductive tract, and agents that increase testicular testosterone concentration agents, are as previously described.

The effective amount of an anti-oxidant agent, and the agent that reduces inflammation in the male reproductive tract and/or the effective amount of an agent that increases testicular testosterone concentration, is not particularly limited, so long as it has the desired or therapeutic effect, and will depend upon the agents administered. Suitable concentrations for the anti-oxidants and other agents are as described previously herein.

The anti-oxidant agent, and the agent that reduces inflammation in the male reproductive tract and/or the agent that increases testicular testosterone concentration, may be administered to the subject separately or in combination.

An effective amount of the anti-oxidant agent and the other agents may be appropriately chosen, depending upon, for example, the type and extent of reduced fertility to be treated, the age and body weight of the subject, the frequency of administration, and the presence of other active agents.

Accordingly, in another embodiment the present invention provides a combination product for improving sperm motility in a male subject, the combination product including the following components:
an anti-oxidant agent; and
an agent that reduces inflammation in the male reproductive tract; and/or
an agent that increases testicular testosterone concentration;
   wherein the said components in the combination product are not the same, and the components are provided in a form for separate administration to the subject, or in a form for co-administration of one or more of the components to the subject.

In one embodiment the anti-oxidant agent, and the agent that reduces inflammation in the male reproductive tract and/or the agent that increases testicular testosterone concentration, are administered to the subject as a composition in the form of a single formulation.

Accordingly, in another embodiment the present invention provides a composition for improving sperm motility in a male subject, the composition including:
(i) an effective amount of an anti-oxidant agent; and
(ii) an effective amount of an agent that reduces inflammation in the male reproductive tract and/or an effective amount of an agent that increases testicular testosterone concentration.

In another embodiment, the present invention provides the use of an anti-oxidant agent, in combination with an agent that reduces inflammation in the male reproductive tract and/or an agent that increases testicular testosterone concentration, in the preparation of a medicament for improving sperm motility in a male subject.

Suitable compositions are as previously described herein, namely the OSMI and Menevit formulations.

The administration of the anti-oxidant agent, and the administration of the agent that reduces inflammation in the male reproductive tract and/or the agent that increases testicular testosterone concentration, are as previously described herein.

The present invention is also suitable for reducing free radical damage to DNA carried by a sperm in a male subject.

Accordingly, in another embodiment the present invention provides a method of reducing free radical damage to sperm DNA in a male subject, the method including the steps of administering to the male subject:
(i) an effective amount of an anti-oxidant agent; and
(ii) an effective amount of an agent that reduces inflammation in the male reproductive tract and/or an effective amount of an agent that increases testicular testosterone concentration.

Methods for determining damage to sperm DNA motility are known in the art. For example, the Sperm Chromatin Structure Assay (SCSA), Comet and the Tunel assay, may all be used to determine damage to sperm DNA as described in Evenson et al (2002) J. Andrology 23(1):25-43 and Shen et al (2000) Free Radical Biol Med 28(4):529-36.

Examples of suitable anti-oxidant agents are as previously described herein. Examples of agents that reduce inflammation in the male reproductive tract, and agents that increase testicular testosterone concentration, are as previously described herein.

The effective amount of the anti-oxidant and other agents is not particularly limited, so long as it has the desired or therapeutic effect, and will depend upon the particular agents administered. Suitable concentrations for the anti-oxidant agents and other agents are as described previously.

The anti-oxidant agent, and the agent that reduces inflammation in the male reproductive tract and/or the agent that increases testicular testosterone concentration, may be administered to the subject separately or in combination.

In one embodiment, this form of the present invention does not involve the administration of an agent that increases testicular concentration.

Accordingly, in another embodiment the present invention provides a method of reducing free radical damage to sperm DNA in a male subject, the method including the steps of administering to the male subject:
(i) an effective amount of an anti-oxidant agent; and
(ii) an effective amount of an agent that reduces inflammation in the male reproductive tract.

An effective amount of the anti-oxidant agent and the other agents may be appropriately chosen, depending upon, for example, the type and extent of reduced fertility to be treated, the age and body weight of the subject, the frequency of administration, and the presence of other active agents.

Accordingly, in another embodiment the present invention provides a combination product for reducing free radical damage to sperm DNA in a male subject, the combination product including the following components:
an anti-oxidant agent; and
an agent that reduces inflammation in the male reproductive tract; and/or
an agent that increases testicular testosterone concentration;
   wherein the said components in the combination product are not the same, and the components are provided in a form for separate administration to the subject, or in a form for co-administration of one or more of the components to the subject.

In one embodiment, the anti-oxidant and the agent that reduces inflammation in the male reproductive tract and/or the agent that increases testicular testosterone concentration are administered to the subject as a composition in the form of a single formulation.

Accordingly, in another embodiment the present invention provides a composition for reducing free radical damage to sperm DNA in a male subject, the composition including:
(i) an effective amount of an anti-oxidant agent; and
(ii) an effective amount of an agent that reduces inflammation in the male reproductive tract and/or an effective amount of an agent that increases testicular testosterone concentration.

In one embodiment, the composition does not include an agent that increases testicular concentration.

Accordingly, in another embodiment the present invention provides a composition for reducing free radical damage to sperm DNA in a male subject, the composition including:
(i) an effective amount of an anti-oxidant agent; and
(ii) an effective amount of an agent that reduces inflammation in the male reproductive tract.

In another embodiment, the present invention also provides the use of an anti-oxidant agent, in combination with an agent that reduces inflammation in the male reproductive tract and/or an agent that increases testicular testosterone concentration, in the preparation of a medicament for reducing free radical damage to sperm DNA in a male subject.

Suitable compositions are as previously described herein, namely the OSMI and Menevit formulations.

The administration of the anti-oxidant agent, and the administration of the agent that reduces inflammation in the male reproductive tract and/or the agent that increases testicular testosterone concentration, are as previously described herein.

The present invention is also suitable for improving sperm production in a male subject.

Accordingly, in another embodiment the present invention provides a method of improving sperm production in a male subject, the method including the steps of administering to the male subject:
(i) an effective amount of an anti-oxidant agent; and
(ii) an effective amount of an agent that reduces inflammation in the male reproductive tract and/or an effective amount of an agent that increases testicular testosterone concentration.

Methods for determining sperm production are known in the art.

Examples of suitable anti-oxidant agents are as previously described. Examples of agents that reduce inflammation in the male reproductive tract, and agents that increase testicular testosterone concentration agents, are as previously described herein.

The effective amount of an anti-oxidant agent, and the agent that reduces inflammation in the male reproductive tract and/or the agent that increases testicular testosterone concentration agents is not particularly limited, so long as it has the desired or therapeutic effect, and will depend upon the particular agents administered. Suitable concentrations for the anti-oxidant and other agents are as described previously herein.

The anti-oxidant agent, and the agent that reduces inflammation in the male reproductive tract and/or the agent that increases testicular testosterone concentration, may be administered to the subject separately or in combination.

An effective amount of the anti-oxidant agent and the other agents may be appropriately chosen, depending upon, for example, the type and extent of reduced fertility to be treated, the age and body weight of the subject, the frequency of administration, and the presence of other active agents.

Accordingly, in another embodiment the present invention provides a combination product for improving sperm production in a male subject, the combination product including the following components:
an anti-oxidant agent; and
an agent that reduces inflammation in the male reproductive tract; and/or
an agent that increases testicular testosterone concentration;
   wherein the said components in the combination product are not the same, and the components are provided in a form for separate administration to the subject, or in a form for co-administration of one or more of the components to the subject.

In one embodiment, the anti-oxidant agent and the agent that reduces inflammation in the male reproductive tract and/or the agent that increases testicular testosterone concentration are administered to the subject as a composition in the form of a single formulation.

Accordingly, in another form the present invention provides a composition for improving sperm production in a male subject, the composition including:
(i) an effective amount of an anti-oxidant agent; and
(ii) an effective amount of an agent that reduces inflammation in the male reproductive tract and/or an effective amount of an agent that increases testicular testosterone concentration.

In another form, the present invention provides the use of an anti-oxidant agent, in combination with an agent that reduces inflammation in the male reproductive tract and/or an agent that increases testicular testosterone concentration, in the preparation of a medicament for improving sperm production in a male subject.

Suitable compositions are as previously described, namely the OSMI and Menevit formulations.

The administration of the anti-oxidant agent, and the administration of the agent that reduces inflammation in the male reproductive tract and/or the agent that increases testicular testosterone concentration, are as previously described.

The present invention is also suitable for improving embryo quality in an embryo produced by fertilization of an oocyte by a sperm from a male subject treated according to the present invention.

Accordingly, in another form the present invention provides a method of improving quality of an embryo produced by fertilization of an oocyte by a sperm from a male subject, the method including the steps of administering to the subject:
(i) an effective amount of an anti-oxidant agent; and
(ii) an effective amount of an agent that reduces inflammation in the male reproductive tract and/or an effective amount of an agent that increases testicular testosterone concentration.

In this regard, the term “embryo quality” will be understood to be a measure of the potential of an embryo to form a viable pregnancy. Embryo morphology is usually considered the best indicator of its quality. On day 2-3 of embryo creation, morphological features such as the number of blastomeres within the embryo, their relative size to one another, degree of cytoplasmic fragmentation and nuclear morphology are all considered good indicators of pregnancy potential (as described in Rienzi et al (2005) Reproductive Biomedicine Online 10(5):669). Furthermore, the ability to progress from the cleavage stage (2-3 days old) to blastocyst (day 5 embryo) is considered a marker of good embryo quality (as described in Borini et al (2005) Reproductive Biomedicine Online 10(5): 653-658.

The embryo may be an embryo produced by a natural conception or an embryo produced by an assisted reproduction technology, such as artificial insemination, in vitro fertilization, gamete intrafallopian transfer (GIFT), intra-uterine insemination (IUI), intracytoplasmic sperm injection (ICSI), testicular sperm extraction (TESE), and percutanenous epididymal sperm aspiration (PESA). Methods for producing embryos are known in the art.

The present invention also provides for an isolated embryo produced by this method, and a non-human animal arising from the embryo.

The embryo with improved quality so produced are also likely to better resist the effects of freezing and thawing. Thus, the present invention also extends to a method of improving the cryopreservation of embryos by fertilizing an oocyte by a sperm isolated from a male subject treated with an effective amount of an anti-oxidant agent, and an effective amount of an agent that reduces inflammation in the male reproductive tract and/or an effective amount of an agent that increases testicular testosterone concentration.

The embryo may be a human embryo, or a mammal embryo such as an embryo from a primate, a livestock animal (eg. a horses, a cow, a sheep, a pig, a goat), a companion animal (eg. a dog, a cat), or a laboratory test animal (eg. a mouse, a rat, a guinea pig). In one embodiment, the embryo is a human embryo.

Accordingly, it will be appreciated that this form of the present may be used in humans and animals to improve embryo quality for natural conception purposes and for assisted reproduction purposes.

Methods for determining embryo quality are known in the art, and are as discussed previously.

Examples of suitable anti-oxidant agents are as previously described. Examples of agents that reduce inflammation in the male reproductive tract, and agents that increase testicular testosterone concentration, are as previously described.

The effective amount of an anti-oxidant agent and the other agents is not particularly limited, so long as it has the desired effect of improving embryo quality, and will depend upon the particular agents administered. Suitable concentrations for the anti-oxidant agent and the other agents are as described previously.

The anti-oxidant agent, and the agent that reduces inflammation in the male reproductive tract and/or the agent that increases testicular testosterone concentration, may be administered to the subject separately or in combination.

An effective amount of the anti-oxidant agent and the other agents may be appropriately chosen, depending upon, for example, the age and body weight of the subject, the frequency of administration, and the presence of other active agents.

Accordingly, in another embodiment the present invention provides a combination product for administration to a male subject to improve quality of an embryo produced by fertilization of an oocyte by a sperm from the male subject, the combination product including the following components:
an anti-oxidant agent; and
an agent that reduces inflammation in the male reproductive tract; and/or
an agent that increases testicular testosterone concentration;
   wherein the said components in the combination product are not the same, and the components are provided in a form for separate administration to the subject, or in a form for co-administration of one or more of the components to the subject.

In one embodiment, the anti-oxidant agent and the agent that reduces inflammation in the male reproductive tract and/or the agent that increases testicular testosterone concentration are administered to the subject as a composition in the form of a single formulation.

Accordingly, in another form the present invention provides a composition for administration to a male subject to improve quality of an embryo produced by fertilization of an oocyte by a sperm from the male subject, the composition including:
(i) an effective amount of an anti-oxidant agent; and
(ii) an effective amount of an agent that reduces inflammation in the male reproductive tract and/or an effective amount of an agent that increases testicular testosterone concentration.

In another form, the present invention also provides the use of an anti-oxidant agent, in combination with an agent that reduces inflammation in the male reproductive tract and/or an agent that increases testicular testosterone concentration, in the preparation of a medicament for administration to a male subject to improve quality of an embryo produced by fertilization of an oocyte by a sperm from the male subject.

Suitable compositions are as previously described, namely the OSMI and Menevit formulations.

The administration of the anti-oxidant agent, and the administration of the agent that reduces inflammation in the male reproductive tract and/or the agent that increases testicular testosterone concentration, are as previously described.

The present invention is also suitable for improving development of an embryo produced by fertilization of an oocyte by a sperm from a male subject.

Accordingly, in another embodiment the present invention provides a method of improving development of an embryo produced by fertilization of an oocyte by a sperm from a male subject, the method including the steps of administering to the subject:
(i) an effective amount of an anti-oxidant agent; and
(ii) an effective amount of an agent that reduces inflammation in the male reproductive tract and/or an effective amount of an agent that increases testicular testosterone concentration.

The embryo may be an embryo produced by a natural conception or an embryo produced by an assisted reproduction technology, such as artificial insemination, in vitro fertilization, gamete intrafallopian transfer (GIFT), intra-uterine insemination (IUI), intracytoplasmic sperm injection (ICSI), testicular sperm extraction (TESE), and percutanenous epididymal sperm aspiration (PESA). Methods for producing embryos by an assisted reproduction technology are known in the art.

The present invention also provides an isolated embryo produced according to this current form of the present invention.

The embryo may be a human embryo, or a mammal embryo such as an embryo from a primate, a livestock animal (eg. a horse, cow, a sheep, a pig, a goat), a companion animal (eg. a dog, a cat), or a laboratory test animal (eg. a mouse, a rat, a guinea pig). In one embodiment, the embryo is a human embryo.

Accordingly, it will be appreciated that this form of the present may be used in humans and animals to improve embryo development for natural conception purposes and for assisted reproduction purposes.

Methods for assessing embryo development are known in the art, and as are discussed previously in relation to embryo quality.

Examples of suitable anti-oxidant agents are as previously described. Examples of agents that reduce inflammation in the male reproductive tract, and agents that increase testicular testosterone concentration, are as previously described.

The effective amount of an anti-oxidant agent and the other agents is not particularly limited, so long as it has the desired effect of improving embryo development, and will depend upon the particular agents administered. Suitable concentrations for the anti-oxidant agent and the other agents are as described previously.

The anti-oxidant agent, and the agent that reduces inflammation in the male reproductive tract and/or the agent that increases testicular testosterone concentration, may be administered to the subject separately or in combination.

An effective amount of the anti-oxidant agent and the other agents may be appropriately chosen, depending upon, for example, the age and body weight of the subject, the frequency of administration, and the presence of other active agents.

Accordingly, in another form the present invention provides a combination product for administration to a male subject to improve development of an embryo produced by fertilization of an oocyte by a sperm from the male subject, the combination product including the following components:
an anti-oxidant agent; and
an agent that reduces inflammation in the male reproductive tract; and/or
an agent that increases testicular testosterone concentration;
   wherein the components in the combination product are not the same, and the components are provided in a form for separate administration to the subject, or in a form for co-administration of one or more of the components to the subject.

In one embodiment, the anti-oxidant agent and the agent that reduces inflammation in the male reproductive tract and/or the agent that increases testicular testosterone concentration are administered to the subject as a composition in the form of a single formulation.

Accordingly, in another form the present invention provides a composition for administration to a male subject to improve development of an embryo produced by fertilization of an oocyte by a sperm from the male subject, the composition including:

(i) an effective amount of an anti-oxidant agent; and

(ii) an effective amount of an agent that reduces inflammation in the male reproductive tract and/or an effective amount of an agent that increases testicular testosterone concentration.

Suitable compositions are as previously described, namely the OSMI and Menevit formulations.

In another form, the present invention also provides the use of an anti-oxidant agent, in combination with an agent that reduces inflammation in the male reproductive tract and/or an agent that increases testicular testosterone concentration, in the preparation of a medicament for administration to a male subject to improve development of an embryo produced by fertilization of an oocyte by a sperm from the male subject.

The administration of the anti-oxidant agent, and the administration of the agent that reduces inflammation in the male reproductive tract and/or the agent that increases testicular testosterone concentration, are as previously described.

The present invention is also suitable for reducing the extent of DNA damage in a subject due to free radical damage to sperm DNA in the father of the subject.

Accordingly, in another form the present invention provides a method of reducing the extent of DNA damage in a subject inherited from the father of the subject, the DNA damage being due to free radical damage to sperm DNA in the father of the subject, the method including the steps of administering to the father prior to conception of the subject:
(i) an effective amount of an anti-oxidant agent; and
(ii) an effective amount of an agent that reduces inflammation in the male reproductive tract and/or an effective amount of an agent that increases testicular testosterone concentration.

This form of the present invention is useful for reducing DNA damage in the progeny of a male subject. The progeny may be produced by natural reproduction or an assisted reproduction technology, such as artificial insemination, in vitro fertilization, gamete intrafallopian transfer (GIFT), intra-uterine insemination (IUI), intracytoplasmic sperm injection (ICSI), testicular sperm extraction (TESE), and percutanenous epididymal sperm aspiration (PESA).

It will be appreciated that this form of the present may be used in humans and animals to reduce DNA damage in progeny, by administration of the various agents to the subject prior to conception of the progeny.

Accordingly, in another embodiment the present invention provides a method of reducing the extent of DNA damage in progeny of a male subject, the DNA damage in the progeny being due to inheritance of DNA damage in sperm of the male subject due to free radicals, the method including the steps of administering to the male subject prior to conception of the progeny:
(i) an effective amount of an anti-oxidant agent; and
(ii) an effective amount of an agent that reduces inflammation in the male reproductive tract and/or an effective amount of an agent that increases testicular testosterone concentration.

Methods for assessing the extent of DNA damage in subjects are known in the art. For example, the Sperm Chromatin Structure Assay (SCSA), Comet and the Tunel assay, may all be used to determine damage to sperm DNA as described in Evenson et al (2002) J Andrology 23(1):25-43 and Shen et al (2000) Free Radical Biol Med 28(4):529-36.

Examples of suitable anti-oxidant agents are as previously described. Examples of agents that reduce inflammation in the male reproductive tract, and agents that increase testicular testosterone concentration, are as previously described.

The effective amount of the anti-oxidant agent and other agents is not particularly limited, so long as it has the desired effect, and will depend upon the agents administered. Suitable concentrations for the anti-oxidant agent and the other agents are as described previously.

The effective amount of the agent that reduces inflammation in the male reproductive tract and/or the effective amount of the agent that increases testicular testosterone concentration is not particularly limited, so long as it has the desired effect, and will depend upon the particular agents administered.

The anti-oxidant agent, and the agent that reduces inflammation in the male reproductive tract and/or the agent that increases testicular testosterone concentration, may be administered to the subject separately or in combination.

In one embodiment, this form of the present invention does not involve the administration of an agent that increases testicular concentration.

An effective amount of the anti-oxidant and the other agents may be appropriately chosen, depending upon, for example, the age and body weight of the subject, the frequency of administration, and the presence of other active agents.

Accordingly, in another embodiment the present invention provides a combination product for administration to a male subject to reduce the extent of DNA damage in progeny of the male subject due to free radical damage to sperm DNA in the male subject, the combination product including the following components:
an anti-oxidant agent; and
an agent that reduces inflammation in the male reproductive tract; and/or
an agent that increases testicular testosterone concentration;
   wherein the components in the combination product are not the same, and the components are provided in a form for separate administration to the subject, or in a form for co-administration of one or more of the components to the subject.

In one embodiment, the anti-oxidant and the agent that reduces inflammation in the male reproductive tract and/or the agent that increases testicular testosterone concentration are administered to the subject as a composition in the form of a single formulation.

Accordingly, in another embodiment the present invention provides a composition for administration to a male subject to reduce the extent of DNA damage in progeny of the male subject due to free radical damage to sperm DNA in the male subject, the composition including:
(i) an effective amount of an anti-oxidant agent; and
(ii) an effective amount of an agent that reduces inflammation in the male reproductive tract and/or an effective amount of an agent that increases testicular testosterone concentration.

Suitable compositions are as previously described, namely the OSMI and Menevit formulations.

In another embodiment, the present invention also provides the use of an anti-oxidant agent, in combination with an agent that reduces inflammation in the male reproductive tract and/or an agent that increases testicular testosterone concentration, in the preparation of a medicament for administration to a male subject to reduce the extent of DNA damage in progeny of the male subject due to free radical damage to sperm DNA in the male subject.

The administration of the anti-oxidant agent, and the administration of the agent that reduces inflammation in the male reproductive tract and/or administration of the agent that increases testicular testosterone concentration, are as previously described.

The present invention is also suitable for preventing a disease or condition occurring in a subject associated with free radical damage to sperm DNA in the father of the subject. Accordingly, in another embodiment the present invention provides a method of preventing a disease or condition in a subject, the disease or condition associated with DNA damage inherited from the father of the subject due to free radical damage to sperm DNA, the method including the steps of administering to the father of the subject prior to conception of the subject:
(i) an effective amount of an anti-oxidant agent; and
(ii) an effective amount of an agent that reduces inflammation in the male reproductive tract and/or an effective amount of an agent that increases testicular testosterone concentration.

This embodiment of the present invention is useful for preventing a disease or condition in the progeny of a male subject. DNA damage to sperm of the father may result in inheritance by the progeny of that DNA damage (pre-zygotic genetic damage), which may ultimately give rise to, or at least contribute to, to the development of a disease or condition in the progeny. As will be appreciated, the father is treated prior to conception of the progeny.

It will be appreciated that this embodiment of the present may be used to prevent a disease in humans or animals. Examples of diseases and conditions associated with free radical damage to sperm DNA in the father of the subject include various types of cancer, such as acute lymphocytic leukaemia.

In one embodiment, the disease or condition is a childhood cancer, such as a childhood cancer that has an onset before the age of fifteen.

Examples of suitable anti-oxidant agents are as previously described. Examples of agents that reduces inflammation in the male reproductive tract, and agents that increase testicular testosterone concentration, are as previously described.

The effective amount of the anti-oxidant agent and the other agents is not particularly limited, so long as it has the desired effect, and will depend upon the particular agents administered. Suitable concentrations for the anti-oxidant agent and the other agents are as described previously.

The anti-oxidant agent, and the agent that reduces inflammation in the male reproductive tract and/or the agent that increases testicular testosterone concentration, may be administered to the subject separately or in combination.

An effective amount of the anti-oxidant agent and the other agents may be appropriately chosen, depending upon, for example, the age and body weight of the subject, the frequency of administration, and the presence of other active agents.

Accordingly, in another embodiment the present invention provides a combination product for administration to a male subject to prevent a disease or condition occurring in progeny of the male subject, the disease or condition in the progeny being associated with free radical damage to sperm DNA in the male subject, the combination product including the following components:
an anti-oxidant agent; and
an agent that reduces inflammation in the male reproductive tract; and/or
an agent that increases testicular testosterone concentration;
   wherein the said components in the combination product are not the same, and the components are provided in a form for separate administration to the subject, or in a form for co-administration of one or more of the components to the subject.

In one embodiment, the anti-oxidant and the agent that reduces inflammation in the male reproductive tract and/or the agent that increases testicular testosterone concentration are administered to the subject as a composition in the form of a single formulation.

Accordingly, in another embodiment the present invention provides a composition for administration to a father of a subject to prevent a disease or condition in the subject associated with DNA damage inherited from the father of the subject due to free radical damage to sperm DNA, the composition including:
(i) an effective amount of an anti-oxidant agent; and
(ii) an effective amount of an agent that reduces inflammation in the male reproductive tract and/or an effective amount of an agent that increases testicular testosterone concentration.

Suitable compositions are as previously described, namely the OSMI and Menevit formulations.

In another embodiment, the present invention provides the use of an anti-oxidant agent, in combination with an agent that reduces inflammation in the male reproductive tract and/or an an agent that increases testicular testosterone concentration, in the preparation of a medicament for administration to a male subject to prevent a disease or condition occurring in progeny of the male subject, the disease or condition being associated with free radical damage to sperm DNA in the male subject.

The administration of the anti-oxidant agent, and the administration of the agent that reduces inflammation in the male reproductive tract and/or the agent that increases testicular testosterone concentration, are as previously described.

The present invention is also suitable for improving fertility in a male subject.

Accordingly, in another embodiment the present invention provides a method of improving fertility in a male subject, the method including the steps of administering to the male subject:
(i) an effective amount of an anti-oxidant agent; and
(ii) an effective amount of an agent that reduces inflammation in the male reproductive tract and/or an effective amount of an agent that increases testicular testosterone concentration.

It will be appreciated that this method may also be used to treat infertility in a male subject.

Accordingly, in another embodiment the present invention provides a method of treating infertility in a male subject, the method including the steps of administering to the male subject:
(i) an effective amount of an anti-oxidant agent; and
(ii) an effective amount of an agent that reduces inflammation in the male reproductive tract and/or an effective amount of an agent that increases testicular testosterone concentration.

It will be appreciated that this embodiment may be used in humans and animals to improve fertility. In addition, the improvement in fertility relates to an improvement to fertilize an oocyte in vitro or in vivo.

Methods for assessing male fertility are known in the art. Routine IVF (non ICSI) provides an excellent test of the in vitro ability of sperm to fertilise an oocyte. Normal fertilization rates are 60-70%, with lesser rates indicating a problem with sperm or oocyte function. Other in vitro tests of sperm-oocyte fertilizing ability include the sperm-zona pellucida (ZP) binding test and the ZP-induced acrosome reaction test (Liu de et al (2004) Fert Steril 82(5): 1251-630).

Examples of suitable anti-oxidant agents are as previously described. Examples of agents that reduce inflammation in the male reproductive tract, and agents that increase testicular testosterone concentration, are as previously described.

The effective amount of an anti-oxidant agent and the other agents is not particularly limited, so long as it has the desired effect, and will depend upon the particular agents administered. Suitable concentrations for the anti-oxidant agent and the other agents are as described previously.

The anti-oxidant agent, and the agent that reduces inflammation in the male reproductive tract and/or the agent that increases testicular testosterone concentration, may be administered to the subject separately or in combination.

An effective amount of the anti-oxidant agent and the other agents may be appropriately chosen, depending upon, for example, the age and body weight of the subject, the frequency of administration, and the presence of other active agents.

Accordingly, in another form the present invention provides a combination product for administration to a male subject to improve fertility, the combination product including the following components:
an anti-oxidant agent; and
an agent that reduces inflammation in the male reproductive tract; and/or
an agent that increases testicular testosterone concentration;
   wherein the components in the combination product are not the same, and the components are provided in a form for separate administration to the subject, or in a form for co-administration of one or more of the components to the subject.

It will be appreciated that the combination product may also be used to treat infertility in a male subject.

In one embodiment, the anti-oxidant agent and the agent that reduces inflammation in the male reproductive tract and/or the agent that increases testicular testosterone concentration are administered to the subject as a composition in the form of a single formulation.

Accordingly, in another embodiment the present invention provides a composition for improving fertility in a male subject, the composition including:
(i) an effective amount of an anti-oxidant agent; and
(ii) an effective amount of an agent that reduces inflammation in the male reproductive tract and/or an effective amount of an agent that increases testicular testosterone concentration.

It will be appreciated that the composition may also be used to treat infertility in a male subject.

Accordingly, in another embodiment the present invention provides a composition for treating infertility in a male subject, the composition including:
(i) an effective amount of an anti-oxidant agent; and
(ii) an effective amount of an agent that reduces inflammation in the male reproductive tract and/or an effective amount of an agent that increases testicular testosterone concentration.

Suitable compositions are as previously described, namely the OSMI and Menevit formulations.

In another embodiment, the present invention provides the use of an anti-oxidant agent, in combination with an agent that reduces inflammation in the male reproductive tract and/or an agent that increases testicular testosterone concentration, in the preparation of a medicament for improving fertility and/or treating infertility in a male subject.

The administration of the anti-oxidant agent, and the administration of the agent that reduces inflammation in the male reproductive tract and/or the agent that increases testicular testosterone concentration, are as previously described.

It has also been recognized that administration of the anti-oxidant agent, and the administration of the agent that reduces inflammation in the male reproductive tract and/or the agent that increases testicular testosterone concentration may be suitable for increasing testosterone levels in a male subject.

Accordingly, the present invention also provides a method of increasing testosterone concentration in a male subject, the method including the steps of administering to the male subject:
(i) an effective amount of an anti-oxidant agent; and
(ii) an effective amount of an agent that reduces inflammation in the male reproductive tract and/or an effective amount of an agent that increases testicular testosterone concentration.

Examples of suitable anti-oxidant agents are as previously described. Examples of agents that reduce inflammation in the male reproductive tract, and agents that increase testicular testosterone concentration, are as previously described.

The effective amount of the anti-oxidant agent and the other agents is not particularly limited, so long as it has the desired effect, and will depend upon the particular agents administered. Suitable concentrations for the anti-oxidant agent and the other agents are as described previously.

The anti-oxidant agent, and the agent that reduces inflammation in the male reproductive tract and/or the agent that increases testicular testosterone concentration, may be administered to the subject separately or in combination.

An effective amount of the anti-oxidant agent and the other agents may be appropriately chosen, depending upon, for example, the age and body weight of the subject, the frequency of administration, and the presence of other active agents.

Accordingly, the present invention also provides a combination product for administration to a male subject to increase testosterone concentration in the male subject, the combination product including the following components:
an anti-oxidant agent; and
an agent that reduces inflammation in the male reproductive tract; and/or
an agent that increases testicular testosterone concentration;
   wherein the components in the combination product are not the same, and the components are provided in a form for separate administration to the subject, or in a form for co-administration of one or more of the components to the subject.

The anti-oxidant agent and the agent that reduces inflammation in the male reproductive tract and/or the agent that increases testicular testosterone concentration may be administered to the subject as a composition in the form of a single formulation.

Accordingly, the present invention also provides a composition for administration to a male subject to increase testosterone concentration in the male subject, the composition including:
(i) an effective amount of an anti-oxidant agent; and
(ii) an effective amount of an agent that reduces inflammation in the male reproductive tract and/or an effective amount of an agent that increases testicular testosterone concentration.

Suitable compositions for increasing testosterone concentration are as previously described, namely the OSMI and Menevit formulations.

The present invention also provides the use of an anti-oxidant agent, in combination with an agent that reduces inflammation in the male reproductive tract and/or an agent that increases testicular testosterone concentration, in the preparation of a medicament for administration to a male subject to increase testosterone concentration in the male subject.

The administration of the anti-oxidant agent, and the administration of the agent that reduces inflammation in the male reproductive tract and/or the agent that increases testicular testosterone concentration, are as previously described.

### DESCRIPTION OF THE PREFERRED EMBODIMENTS

Reference will now be made to experiments that embody the above general principles of the present invention. However, it is to be understood that the following description is not to limit the generality of the above description.

### Example 1

### Trial to Examine OSMI Nutraceutical in a Group of Male Subjects with Known Free Radical Damage

A small (n=17) pilot study was conducted to examine the usefulness of the OSMI nutraceutical in a group of men with known free radical damage. Infertile men were screened for free radical damage using the TBARS assay as described in Gomez et al (1998) International Journal of Andrology 21(2):81-96). Those men found to have significantly elevated levels of Malondealdehyde (MDA), a marker of sperm membrane oxidation, were enrolled in the trial. All patients received the active OSMI medication (ie no placebo) for a period of 12 weeks.

The OSMI formulation was as follows:

Vitamin E (d-alpha-tocopheryl acetate), 400 I.U.
Vitamin C (ascorbic acid or a salt thereof) 100 mg

### Lycopene 6 mg

### Co-Enzyme Q10 40 mg

### Selenium 26 μg

### Zinc 25 mg

### Garlic Oil 1000 mg

The formulation was provided in a capsule and administered as one capsule orally per day.

During the period of the trial, changes in the sperm count, motility, membrane integrity and DNA damage were analysed (entry, 6 weeks, 12 week time points). In addition changes in MDA levels were monitored to detect any modification of sperm membrane lipid peroxidation as a result of free radical damage.

Sperm count and motility were assessed by assessed by usual lab techniques, as described in detail within the World Health Organisation (WHO) laboratory manual for the examination of human semen and sperm-cervical mucous interaction. 4th edition. Cambridge University Press 1999.

Sperm vitality (membrane integrity) was assessed by the HOST test, which measures the proportion of sperm that have intact sperm membrane, as described in detail within the World Health Organisation (WHO) laboratory manual for the examination of human semen and sperm-cervical mucous interaction. 4th edition. Cambridge University Press 1999.

Damage to sperm DNA was assessed by a DNA fragmentation (TUNEL) assay using an In-Situ Cell Death Detection Kit, Fluorescien (Roche Diagnostics) As described by (Lachaud et al (2004) Hum Reprod 19(3): 607-10.). Briefly, 90% Percoll fractionated and washed sperm are microscope slide smeared and fixed in 4% paraformaldehyde, permeabilised (0.1% triton X, 0.1% Sodium Citrate.) and incubated 37° C. for 1 hour in TUNEL incubation buffer and TdT enzyme terminal transferase, robustly washed in PBS and counterstained with 10 μg/ml nuclear Propidium Iodide.

Slides are then processed using a Nikon TE2000E epi-fluorescent microscope and imaged with a Roper CS CCD camera utilising a FITC filter excitation 465-495 nm, Barrier filter 515-555 nm, Dichroic mirror at 505 nm for apoptotic channel fluorescence and a PI filter excitation 540-625 nm, Barrier filter 605-655 nm Dichroic mirror at 565 nm.

A total of ˜>200 cells are randomly analysed, multiple images captured and TUNEL Green apoptotic nuclear fluorescence is graphically mapped over the Red nuclear PI fluorescence and the overlap positive scores are individually quantitated using Scanalytics IP lab software. A final average percentage of sperm in a population with fragmented DNA is calculated, referred to as a TUNEL % and is reported, generating an average intra assay SEM of <3.

Sperm lipid peroxidation was assessed by the TBARS assay, as described in Gomez et al (1998) International Journal of Andrology 21(2):81-96).

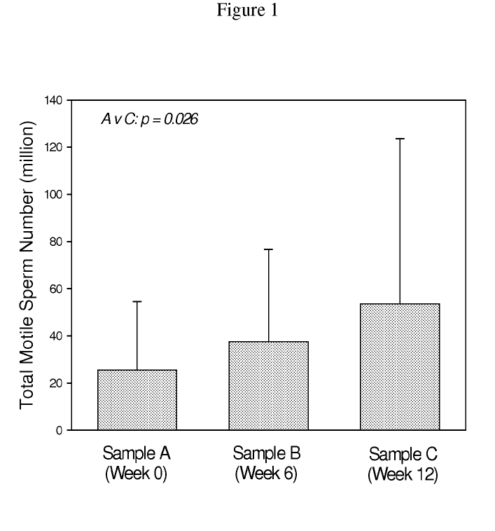

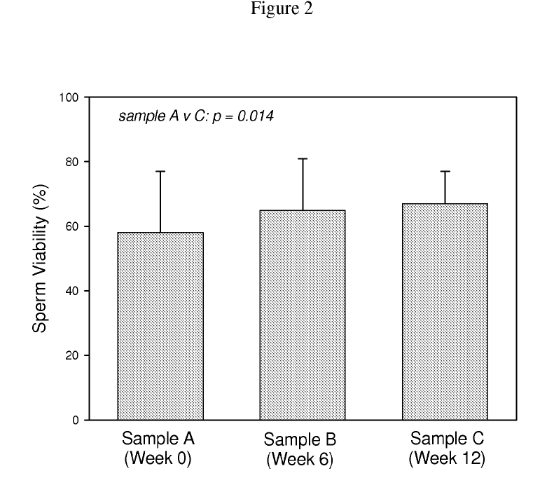

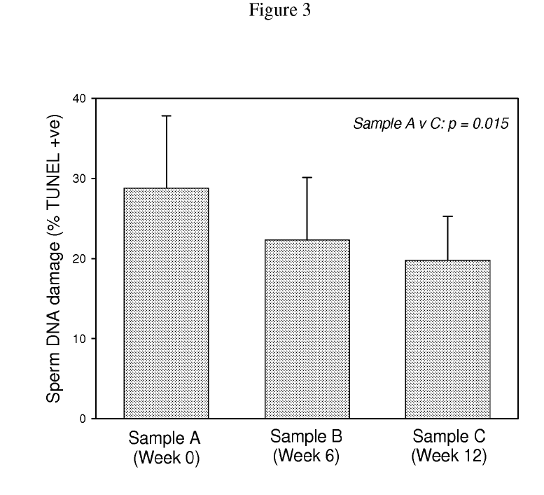

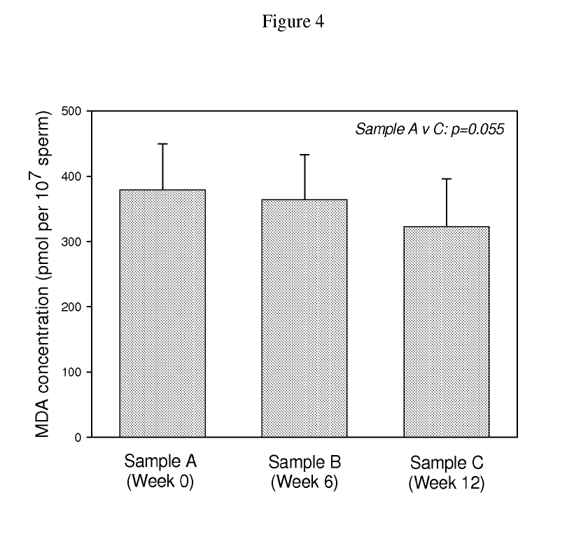

The results of this study by the 12-week mark were as follows:
1. A doubling in the motile sperm count (25.6 million to 53.6 million), as shown in FIG. 1.
2. A significant improvement in sperm vitality as assessed by the HOST test (58% v 67%). The higher the level of free radical damage, the lower the HOST percentage. The data is shown in FIG. 2.
3. A significant fall in sperm DNA damage (28.8% to 19.8%), as shown in FIG. 3.
4. A reduction in MDA, reflecting a decline in free radical sperm membrane lipid peroxidation damage, as shown in FIG. 4.

It is noteworthy that while sperm parameters did improve slightly by the mid-point (6 week) stage of the trial, full beneficial effects took 12 weeks.

### Example 2

### Effect of OSMI Nutraceutical on Pregnancy and IVF Embryo Quality

Pregnancy and IVF embryo quality was not a primary endpoint of the initial OSMI trial. However, several patients did fall pregnant either spontaneously or with IVF assistance. Those patients who had received IVF before and during OSMI treatment provided a measure of how the OSMI nutraceutical could affect embryo quality.

Couple A had previously had multiple cycles of IVF with poor quality embryos (0 out of 8 embryos formed blastocyst in pre-OSMI IVF cycle). However, while on the OSMI nutraceutical three out of 9 embryos progressed to blastocyst and the female partner subsequently became pregnant. Survival of an embryo beyond the third day requires good embryo DNA quality, making blastocyst development a good marker of sperm DNA health.

### Example 3

### A Randomized Control Trial Investigating the Effect of an Anti-Oxidant Medication (Menevit®) on Sperm Function and Pregnancy Outcome During IVF Treatment

Impairment of sperm function accounts for half of all cases of infertility. It is estimated that one in twenty men have impaired sperm function, with an estimated 1.2 million men currently experiencing male related infertility in the United States. Traditionally male infertility treatment has not endeavored to ameliorate the underlying cause of infertility but rather used “mechanical” techniques such as intra-uterine insemination or IVF-ICSI to bypass the defect in sperm function. While these two techniques are undeniably successful in a large proportion of patients, they simply do not work or have very limited efficiency in other couples. It is likely that in many cases, sperm DNA fragmentation is responsible for the poor pregnancy outcome despite ART treatment. Treatments that can prevent sperm DNA fragmentation are likely to boost both natural and ART related pregnancy rates.

The sources of sperm DNA damage have not been fully elucidated. To provide a pharmacotherapeutic route to reduce sperm fragmentation, the Menevit nutraceutical was developed. The contents of Menevit are outlined in Table 1. The current prospective randomized placebo-controlled trial was designed to test this hypothesis.

TABLE 1
Study capsule components

1. Menevit active capsule
Lycopene 6 mg
Vitamin E 400 i.u.
Vitamin C 100 mg
Zinc 25 mg
Selenium 26 μgm
Folate 0.5 mg
Garlic 1000 mg
Palm oil (vehicle)
2. Placebo capsule
Palm oil

### Materials and Methods

Participants for this study were recruited from those couples undergoing IVF treatment at Repromed, The University of Adelaide's Reproductive Medicine Unit. To be eligible for enrollment men had to have likely oxidative related sperm damage signified by either poor motility (Aitken et al 1993) or a poor HOST test result on their entry semen sample, be a smoker (Saleh et al 2002) or have a varicocele (Pasqualotto et al 2000) and significant sperm DNA fragmentation (>25% sperm DNA fragmentation on TUNEL assay). In addition, the partners of these men had to be undergoing a stimulated cycle of IVF within 3 months of enrollment and have normal ovarian reserve. We did not want female factors such as poor ovarian reserve to affect the outcome of the trial so we excluded all women 40 years of age and older, those with a poor prior IVF response (<5 oocytes) or elevated early follicular phase FSH result (>10 iu/l). Recruitment commenced in December 2004 and the trial was complete by April 2006. Before commencement the study was approved by the Women's and Children's Hospital research ethics committee.

Information on demographics, fertility and pregnancy history and prior IVF treatment outcome were collected for all patients, as outlined in Table 2.

Those subjects eligible for enrolment were randomly allocated to the active Menevit nutraceutical or placebo at a ratio of 2:1. This uneven allocation was deemed necessary when pre-trial patient surveys suggested that if participants were offered a 50% chance of receiving active anti-oxidant treatment, many would self supplement with over the counter anti-oxidants. This was deemed less likely with a 2:1 active to placebo allocation. The randomization schedule was computer generated in blocks of six by Bayer Consumer Care Australia, and the appropriately numbered bottles of capsules delivered to the clinical site without any clinical participant knowing the treatment sequence. Patients were allocated the next numerical treatment package (1-60) as they became eligible for enrolment. The active Menevit and placebo were identical in appearance and taste.

Male participants were asked to take one capsule per day after food, starting 3 months prior to their partners IVF oocyte retrieval. All participants were supplied with 4 months of medication in case of delays in their IVF cycle. The men were then asked to provide a semen sample at the 6 and 12 week mark to monitor changes in sperm function. These samples were produced by masturbation after a period of 3-5 days abstinence and analyzed for sperm count, motility and morphology as per WHO guidelines. In addition a Hypo-Osmolar Swelling Test (HOST) was conducted to measure sperm membrane integrity, as outlined in the WHO semen analysis manual. The remaining sample was frozen neat without cryoprotectant for later analysis of sperm DNA fragmentation and oxidative damage.

Sperm DNA fragmentation was assessed using the microscopic TUNEL assay (Lopes et al. (1998) Hum Reprod. 13(4):896-900). Sperm were obtained using density gradient centrifugation (2000 rpm, 20 minutes) through a 45%/90% Percoll density gradient, smeared on polylysine slides, air-dried and fixed with 4% paraformaldehyde in PBS. The sperm were then permeablised with 0.5% Triton X-100, washed with PBS before being incubated with terminal deoxyribonucleotidyl transferase-mediated dUTP nick-end labeling (TUNEL) as per the manufacturers instructions (Roche, Mannhein, Germany). The smear was again washed and the sperm nuclei stained with propidium iodide before fluroscent microscopy assessment. Density gradient centrifugation of the semen sample was critical to remove seminal debris which had auto-fluorescence activity and made microscopic TUNEL assessment difficult. A total of 200 sperm per slide were assessed using image analysis software, with the percentage sperm DNA fragmentation being calculated as the number of TUNEL positive nuclei (green) per total number of sperm nuclei (red). For a positive control sperm were incubated with 3 iu DNAse prior to incubation with the TUNEL mixture and for a negative control the terminal transferase was omitted from the reaction.

The LPO-586 assay for sperm lipid peroxidation was conducted as per the protocol of Gomez et al (1998) Int J Androl. 21(2):81-94 and purchased from Bioxytech SA (Bonneuil sur Marne, France). The LPO-586 assay is based on the reaction of a chromogenic reagent (N-methyl-2phenytindole) with the byproducts of lipid peroxidation, malonaldehyde and 4-hydroxyalkenal, to create a stable chromophore with maximal absorbance at 560 nm. As many sperm samples have low baseline levels of lipid peroxidation, a 0.04 M ferric sulphate ionic promoter was used to improve the assay sensitivity. Sperm concentrations were standardized to 1×10⁶/ml, except in cases where sperm count was less than 1×10⁶/ml in the neat sample. Here mathematical scaling was used to calculate lipid peroxidation levels per 1×10⁶ sperm per ml.

The IVF procedures consisted of a typical long down-regulation protocol with GnRH agonist (naferilin acetate or leuprolide acetate) commencing in the mid-luteal phase of the preceding cycle. At day 2 of the stimulation cycle women were commenced on 150-300 iu of rFSH (Puregon, Organon or Gonal-F, Serono) depending upon their age and previous IVF response. Ovarian response was tracked by pelvic ultrasound and serum estradiol, with 5000 IU hCG (Pregnyl, Organon) being administered when at least two follicles were >18 mm in size with an adequate estradiol response. Trans-vaginal oocyte retrieval was conducted under sedation 36 hours after hCG administration, followed by standard IVF or ICSI fertilization procedures. Cleavage stage embryos were graded according to traditional morphological criteria (blastomere shape, number and percentage fragmentation) and returned to the uterus on day 2 or 3 post oocyte collection under ultrasound guidance. Remaining good quality embryos were frozen on day 3, with any poor quality embryos being cultured out to day 6 before a decision was made to discard. Elective blastocyst culture and transfer was used by a minority of patients in this trial. All patients had luteal support using a combination of Crinone 8% vaginal progesterone (Serono) and a single 500 IU injection of hCG on day 6 post oocyte retrieval. Serum pregnancy tests were performed 16 days after oocyte retrieval in the absence of a menstrual period. First trimester pregnancy scans were conducted at 8 weeks gestation using a 7.5 MHz Toshiba trans-vaginal scanner.

Subject compliance and side effect monitoring was assessed by a questionnaire completed by the male partner on the day of oocyte retrieval. All participants were asked how often, if ever, they missed their medication and whether they noticed any side effects during their treatment. Data was analyzed on an “intention-to-treat” basis, irrespective of male medication compliance.

The primary outcome for this trial was number of good quality embryos generated per IVF cycle, a reasonable surrogate marker of pregnancy potential. Previous observations in our lab had suggested that cleavage stage embryo quality was decreased in those men with high DNA damage. On average in our IVF unit women less than 40 years of age produce 3.6 good quality embryos per IVF cycle. Pilot observations suggested that only 2 good quality embryos were produced by men with high levels of DNA fragmentation. Power analysis was then performed to detect a minimum increase of one good quality embryo (from 2 to 3 good quality embryos) per IVF cycle started. A trial of 60 IVF cycles would detect a clinically significant difference between groups, assuming a power of 80%, two sided testing at the 5% significance level and a 10% IVF drop-out rate. The secondary outcomes included sperm function (count, motility, morphology, HOST result, sperm DNA fragmentation, sperm lipid peroxidation) and IVF outcomes (fertilization rate, embryo quality, pregnancy rates).

Data were analyzed using commercial software (Statistical Package for the Social sciences 11.5.1; SPSS, Chicago, Ill.). Baseline demographic and fertility related variables between groups were analyzed using unpaired t-test for continuous variables and Chi Square for categorical variables. Differences in sperm function within patients during the trial were analyzed by the paired t-test. Differences in embryo quality and pregnancy outcome were analyzed by Chi-Square analysis. A p value <0.05 was considered significant.

### Results

A total of 82 men were screened for entry into the trial, with 22 being excluded due to low levels of sperm DNA damage or no evidence of oxidative stress. Six study participants did not complete the trial due to their decision to withdraw from IVF treatment (2 active arm, 4 placebo). In five of these withdrawals the male continued to take his trial medication and produce semen samples for study analysis. One participant in the active medication arm did not reach an embryo transfer because no embryos were available for transfer due to immediate oocyte lysis at time of ICSI. This woman was on a severe caloric restriction diet at the time of IVF treatment. As she did become pregnant in the next cycle while off the diet (while her husband was still on anti-oxidants but out of the trial), the metabolic alterations of severe dieting were felt to be responsible for oocyte lysis rather than the anti-oxidant treatment. Another active arm participant was unable to have a fresh embryo transfer due to severe ovarian hyper-stimulation. No participant withdrew from the study because of spontaneous conception prior to trial exit. However, two participants on the active Menevit medication did conceive spontaneously within 1 month of exiting the trial (data not included in study analysis).

The baseline characteristics of trial participants are recorded in Table 2.

TABLE 2
Participant demographic and baseline IVF characteristics

| | Active | | |
|---|---|---|---|
| | Menevit | Placebo | P |
| Female age (years) | 34.6 ± 3.4 | 33.6 ± 3.9 | NS |
| Male age (years) | 37.1 ± 5.1 | 35.5 ± 4.3 | NS |
| Duration infertility (years) | 4.2 ± 2.7 | 3.4 ± 2.1 | NS |
| Gravidity | 0.77 ± 0.9 | 0.55 ± 0.8 | NS |
| Etiology of infertility | | | |
| Male | 45% | 50% | NS |
| Combined | 55% | 50% | |
| Prior IVF treatment (%) | 52.5% | 55% | NS |
| Number prior IVF cycles | 1.8 ± 1.9 | 1.5 ± 1.9 | NS |
| Oocytes in prior IVF cycle | 10.5 ± 4.3 | 9.9 ± 2.6 | NS |
| Fertilization rate prior IVF cycle (%) | 56.9% | 57.5% | NS |
| Prior IVF embryo quality (%) | | | |
| Grade 1 (excellent) | 14.7% | 15.8% | NS |
| Grade 2 (good) | 33.7% | 47.3% | |
| Grade 3/4 (poor) | 51.6% | 36.8% | |

Note:
values are mean ± SD.

There were no significant differences between the active and placebo group in terms of important baseline prognostic characteristics such as maternal/paternal age, past reproductive history and etiology of infertility. Furthermore, the group's prior IVF experiences were not significantly different when considering the number of prior IVF cycles, the number of oocytes collected in previous IVF cycles and the resulting embryo quality (Table 2). This would suggest that randomization had been successful in equally distributing the important confounding variables between the two groups.

Pregnancy outcomes were significantly better in the active (Menevit) treatment group compared to the placebo (Table 3).

TABLE 3
Pregnancy outcomes by study group

| | Active | | |
|---|---|---|---|
| | Menevit | Placebo | P |
| Embryo transfer procedures | 36 | 16 | |
| Total number of embryos | 52 | 25 | |
| transferred | | | |
| Biochemical pregnancy | 3 | — | |
| Clinical miscarriage | 2 | 2 | |
| Ectopic pregnancy | 1 | — | |
| Viable Singleton | 13 | 4 | |
| Viable singleton/ | 1 | — | |
| nonviable twin | | | |
| Viable twin | 3 | — | |
| Pregnancy rate | 23/36 (63.9%) | 6/16 (37.5%) | 0.077 |
| (positive βHCG) | | | |
| Implantation rate ^{a} | 24/52 (46.2%) | 6/25 (24%) | 0.062 |
| Viable pregnancy rate ^{b} | 20/52 (38.5%) | 4/25 (16%) | 0.046 |

^{a} Implantation rate calculated as the % of transferred embryos resulting in a clinical pregnancy (gestational sac) on first trimester scan
^{b} Viable pregnancy rate calculated as the % of transferred embryos resulting in a viable fetal heart on first trimester scan.

The Menevit implantation rate was almost double that of the placebo (46.2% v 24%, p=0.06), with the differences in viable fetal hearts at 13 weeks gestation (38.5% v 16%) being statistically significant. The baseline implantation rate for women under 38 years of age at Repromed (2005, n=709 transfer procedures) was 35%, with only 7% having two or more gestational sacs. This low multiple pregnancy rate is due to an almost universal policy of single embryo transfer in women under 36 years of age in their first 2 cycles of IVF. In our study the implantation rate was significantly higher than the general IVF population as 4 of the women in the Menevit group had twin gestational sacs on first trimester scan (8 from 25 sacs in total were twin sacs-32%). The high twin gestational sac rate was not due to a higher than average number of embryos being transferred per cycle. A mean number of only 1.39 embryos were transferred in the active Menevit group which was not significantly different to the Repromed average of 1.3 embryos per transfer in women under 38 years. Therefore it is likely that the embryos transferred in the active Menevit group had a higher implantation potential then either the embryos derived from the placebo arm of this study or the general non-trial IVF population.

It is uncertain why embryos from the Menevit group had a higher implantation potential compared to the placebo group as there was no discernable difference in the cleavage stage embryo quality (Table 4). Differences in embryo quality may have been detected if extended culture to blastocyst had been performed. However this analysis was not possible as blastocyst culture was only used in a small proportion of trial patients.

TABLE 4
IVF cycle outcomes by study group

| | Active | | |
|---|---|---|---|
| | Menevit | Placebo | P |
| Number of oocytes collected | 11.4 ± 4.4 | 9.6 ± 3.9 | 0.15 |
| Metaphase II oocytes injected | 9.3 ± 3.8 | 7.9 ± 3.2 | 0.19 |
| Fertilization rate (%) | 68.8% | 63.0% | NS |
| Embryo quality | | | |
| Grade 1 (excellent) | 11.6% | 13.7% | |
| Grade 2 (good) | 44.2% | 37.6% | |
| Grade 3/4 (poor) | 44.2% | 48.7% | |
| Embryos transferred | 1.39 ± 0.6 | 1.56 ± 0.5 | 0.33 |
| Embryos cryo-preserved | 1.71 ± 0.5 | 1.40 ± 0.5 | 0.32 |

Note.
Values are mean ± SD.

Analysis of the effect of the Menevit nutraceutical on general sperm parameters showed that it had no significant effect on sperm concentration, motility or morphology (Table 5). Furthermore, neither the LPO-586 assay for lipid peroxidation damage nor the sperm membrane integrity test (HOST) could detect any significant difference between the two study groups in levels of free radical damage to the sperm membrane. The HOST results of both the placebo and Menevit group showed a very small but statistically significant improvement over time. These differences were equal between the two study groups and very small in absolute terms. As a low HOST result was often used as a criteria for inclusion (evidence of sperm free radical damage) this small improvement in HOST scores is likely to reflect statistical “regression to the mean” rather than a true biological effect.

Changes in sperm DNA fragmentation during the trial are outlined in Tables 5 and 6.

TABLE 5
Sperm parameters in Menevit group

| | Menevit active | | |
|---|---|---|---|
| | (n = 39) | | |
| | Entry | 6 week | 12 week |
| Sperm concentration | 26.1 + 26.4 | 22.2 + 22.9 | 28.6 + 36.2 |
| (×10⁶/ml) | | | |
| Sperm motility (%) | 32.1 + 15.9 | 33.7 + 17.4 | 32.2 + 17.1 |
| Normal sperm | 5.1 + 4.1 | 5.4 + 4.8 | 5.5 + 5.2 |
| morphology (%) | | | |
| Sperm vitality (%)- | 55.5 + 12.6 | 59.2 + 12.8 | 60.5 + 12.0^{a} |
| HOST | | | |
| Sperm DNA | 37.9 + 11.9 | 33.5 + 11.6^{a} | 33.3 + 12.3 |
| fragmentation (%) | | | |
| Sperm lipid | 2.3 + 1.3 | 2.5 + 1.3 | 2.4 + 1.2 |
| peroxidation (μmol) | | | |

Note:
Data are mean + SD
^{a}p ≦ 0.05 for comparison between baseline and post-treatment values (paired t-test)

TABLE 6
Sperm parameters in Placebo group

| | Placebo | | |
|---|---|---|---|
| | (n = 20) | | |
| | Entry | 6 week | 12 week |
| Sperm concentration | 26.7 + 27.5 | 23.1 + 22.3 | 24.8 + 22.8 |
| (×10⁶/ml) | | | |
| Sperm motility (%) | 36.4 + 13.8 | 41.7 + 15.8^{a} | 38.8 + 16.1 |
| Normal sperm | 6.8 + 4.4 | 5.9 + 5.2 | 6.8 + 5.8 |
| morphology (%) | | | |
| Sperm vitality (%)- | 56.0 + 18.7 | 62.9 + 17.3^{a} | 60.1 + 19.6 |
| HOST | | | |
| Sperm DNA | 40.3 + 15.3 | 31.4 + 15.7^{a} | 32.0 + 12.0^{a} |
| fragmentation (%) | | | |
| Sperm lipid | 2.5 + 1.2 | 2.3 + 0.7 | 2.3 + 0.6 |
| peroxidation (μmol) | | | |

Note:
Data are mean + SD
^{a}p ≦ 0.05 for comparison between baseline and post-treatment values (paired t-test)

It is interesting to note that sperm DNA damage was reduced in both the active Menevit and placebo groups, with no significant difference being noted between the two groups. The fall in sperm DNA fragmentation in both groups suggest that any improvement in sperm DNA was due to statistical regression to the mean rather than a true biological response.

A total of 59 men completed a minimum of 12 weeks of “medication” and 55 completed a side effects questionnaire (93% return rate). Compliance with taking the medication during the entire trial was excellent with 96% of participants missing less than 1 capsule per week. None of the men on the placebo noted any side effects. In the Menevit group 3 of the 37 men (8%) who returned the questionnaire noted mild side effects. Two of these reported side effects were mild gastro-esophageal reflux and the other constipation. No participant felt that the side effects were significant enough to consider withdrawing from the trial.

### Discussion

To the best of our knowledge this study is the first randomized control trial (RCT) showing that an anti-oxidant preparation can boost pregnancy rates during IVF treatment.

The magnitude of improvement in pregnancy rates in this trial far exceeded our expectations. When designing the study we did not choose pregnancy as the primary outcome as power calculations suggested it would have required a very large study for the traditional 25% minimum clinical improvement. Instead, cleavage stage embryo quality was used as a marker of improved pregnancy potential. Cleavage stage embryo quality is correlated with pregnancy potential and prior studies had shown that men with high degrees of sperm DNA damage have inferior cleavage stage embryo morphology compared to those men with low levels of DNA damage. Our study was unable to detect any significant effect of anti-oxidant medication on cleavage stage embryo quality that could help explain the observed improvements in pregnancy rates. Blastocyst culture is probably a better marker of sperm DNA integrity than cleavage stage assessment. Unfortunately we were unable to analyze blastocyst development rates in our trial as it was not common clinical practice in our unit to perform extended culture for women under 40 years of age.

The Menevit anti-oxidant treatment had no significant effect on sperm count, motility or morphology. The present study also did not confirm the ability of anti-oxidant to reduce sperm DNA damage compared to the placebo.

When it became apparent that the large improvement in pregnancy outcome from anti-oxidant supplementation was not linked with an improvement in sperm DNA fragmentation, we analyzed the correlation between the 12 week DNA fragmentation results and pregnancy outcome. Surprisingly we found there was absolutely no link between the overall 12 week TUNEL results and pregnancy outcome (viable pregnancy=36%+10% DNA fragmentation, no viable pregnancy=31%+13%). This was not expected as previous work within our laboratory analyzing sperm DNA fragmentation in the semen sample used for oocyte insemination had shown a significant negative correlation between the TUNEL result and pregnancy outcome. In the present study we did not perform sperm DNA fragmentation studies on the semen sample used for insemination as it was felt that the study assays would have consumed most of the sample, leaving little for clinical use. As there was on average a further 3 weeks of anti-oxidant treatment before production of a semen sample for IVF use, it is possible that improvements in sperm DNA may have been present in this later clinical sperm sample, thereby explaining the increase in pregnancy rates.

Two final problems when trying to interpret sperm DNA fragmentation levels and pregnancy outcome is density gradient “normalization” and the IVF-ICSI “iceberg phenomenon” (Makhlouf and Niederberger (2006) J Androl. 27(3):316-23). All of the patients within the current study had sperm for fertilization prepared using density gradient centrifugation. This type of sperm processing has a “normalizing effect”, as the sperm in the highest density layer used for fertilization are usually of very good quality, irrespective of the overall general sperm population's quality before gradient centrifugation. Two studies have shown that density gradient centrifugation improves sperm DNA integrity results in TUNEL analysis by 2 to 4 fold (Tomlinson et al (2001) Hum Reprod. 16(10):2160-5); Morrell et al (2004) J Assist Reprod Genet. 21(6):217-22). Therefore it is possible that anti-oxidant treatment did improve sperm DNA quality in the neat sample, but this difference was no longer apparent after “normalizing” using density gradient centrifugation. Finally, during ICSI treatment only a few top quality sperm are used for fertilization, with the remaining millions being discarded (“iceberg phenomenon”). It is therefore possible that an anti-oxidant treatment may improve the DNA quality of these top quality sperm that have the least amount of baseline damage, but this improvement is lost in the overall analysis because of no significant effect on the DNA damage of the remaining 99.9% of sperm not used for fertilization.

The present study is the first double-blind placebo controlled randomized study to show that an antioxidant nutraceutical (Menevit) has the ability to boost pregnancy rates during IVF treatment. The mechanism by which this occurs is presently unclear. However we believe it is most likely to be mediated by improvements in sperm DNA damage, despite our inability to detect such improvements, for the many potential reasons outlined in the previous discussion. Future studies examining the effect of the Menevit nutraceutical using more sensitive assays will hopefully shed light on the mechanisms of improvement in pregnancy rates. We do acknowledge that our study of 60 patients is only relatively small and that the observed improvement in pregnancy rates could be a “statistical fluke” (type 1 statistical error). However, the occurrence of several “miracle” pregnancies amongst our long term IVF patients while on anti-oxidant treatment suggests that the observed significant improvement in pregnancy rates is a real biological phenomenon, not a statistical anomaly.

Any new medication should be assessed by three principal criteria: clinical effectiveness, cost and side effect profile. This study has shown that the Menevit nutraceutical is effective in boosting pregnancy rates during IVF treatment, without altering basic sperm parameters. Finally, the Menevit nutraceutical was free of any severe side effects, with only a minority of patients experiencing mild gastro-intestinal side effects.

Finally, it will be appreciated that various modifications and variations of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are apparent to those skilled in the art are intended to be within the scope of the present invention.

1-83. (canceled)

84. A method for improving reproductive health in a male subject, the method comprising administering to the male subject:
(i) an effective amount of at least one anti-oxidant agent;
(ii) an effective amount of at least one agent that reduces inflammation in the male reproductive tract and/or increases testicular testosterone concentration; and
(iii) an effective amount of at least one agent that improves sperm function and/or is involved in cellular DNA synthesis,
wherein the at least one agent that reduces inflammation in the male reproductive tract and/or increases testicular testosterone concentration comprises garlic or an extract, oil or active compound derived therefrom.

85. A method of claim 84 wherein improving reproductive health comprises, or is associated with, one or more of the following: reducing free radical damage to sperm; reducing the generation of free radicals in the reproductive tract and/or semen; improving sperm function; improving sperm motility; improving sperm production; reducing free radical damage to sperm DNA; improving fertility; preventing and/or treating infertility; increasing testosterone concentration; reducing activity and/or concentration of leukocytes in the reproductive tract and/or semen; and reducing inflammatory cytokine production in the reproductive tract and/or semen.

86. The method of claim 84 wherein the at least one anti-oxidant agent is selected from one or more of the group consisting of a β-carotenoid, comprising lycopene, lutein, and zeaxanthin; Vitamin C; Vitamin E; Co-Enzyme Q10; selenium; zinc; L-carnitine; acetylcarnitine; N-acetylcysteine; glutathionine; pyruvate; and hypotaurine.

87. The method of claim 84 wherein the at least one agent that reduces inflammation in the male reproductive tract further comprises one or more of the group consisting of green tea, or an extract or active compound derived therefrom; N-3 fatty acids, docosahexaenoic acid, ginger or an extract or active compound derived therefrom;
an agent that decreases leukocyte production of TNFα, comprising pentoxyphylline; an agent that blocks the action of TNFαonce produced, comprising infliximab; and lipid extract from marine mollusk.

88. The method of claim 84 wherein the at least one agent that increases testicular testosterone concentration further comprises one or more of the group consisting of zinc; tribulus terrestris or an extract or active compound derived therefrom; an agent that reduces inducible nitric oxide synthase in macrophages; and an agent that reduces nitric oxide production.

89. The method of claim 84 wherein the administration to the subject comprises daily administration of about 0.5 to 50 mg lycopene, about 10 to 1000 mg Vitamin C, about 40 to 4000 I.U. Vitamin E, about 10 to 250 μg selenium, about 2.5 to 100 mg zinc, at least about 500 mg garlic, and optionally about 4 to 400 mg Co-Enzyme Q10.

90. The method of claim 84 wherein the administration to the subject comprises daily administration of about 2 to 10 mg lycopene, about 20 to 200 mg Vitamin C, about 200 to 600 I.U. Vitamin E, about 20 to 50 μg selenium, about 10 to 50 mg zinc, at least about 500 mg garlic, and optionally about 10 to 100 mg Co-Enzyme Q10.

91. The method of claim 84 wherein the administration to the subject comprises daily administration of garlic oil equivalent to about 1000 mg garlic bulb.

92. The method of claim 84 wherein the administration to the subject comprises daily administration of about 6 mg lycopene, about 100 mg Vitamin C, about 400 I.U. Vitamin E, about 26 μg selenium, about 25 mg zinc, about 1000 mg garlic, and optionally about 40 mg Co-Enzyme Q10.

93. The method of claim 84 wherein administration to a subject is for a period of at least 10 weeks.

94. The method of claim 84 wherein the at least one agent that improves sperm function and/or is involved in cellular DNA synthesis is folic acid or a salt thereof.

95. The method of claim 84 wherein the method comprises the administration of about 500 μg folate.

96. The method of claim 84 wherein the male subject is selected from the group consisting of a subject with increased levels of sperm membrane oxidation, comprising a subject with increased levels of malondialdehyde or other biochemical markers of oxidative stress; a smoker; a subject with reduced fertility, comprising reduced fertility due to poor sperm motility, or reduced fertility of unknown origin; a subject having undergone vasectomy reversal; a subject with a reproductive tract infection such as epididymitis; and a subject having a varicocele.

97. The method of claim 84 wherein the administration of at least one of said agents is administered to the subject by oral administration.

98. A method for improving pregnancy outcome in a female subject, the female subject or an oocyte introduced into the female subject being fertilized by a sperm from a male subject, the method comprising administering to the male subject prior to fertilization:
(i) an effective amount of at least one anti-oxidant agent;
(ii) an effective amount of at least one agent that reduces inflammation in the male reproductive tract and/or increases testicular testosterone concentration; and
(iii) an effective amount of at least one agent that improves sperm function and/or is involved in cellular DNA synthesis,
wherein the at least one agent that reduces inflammation in the male reproductive tract and/or increases testicular testosterone concentration comprises garlic or an extract, oil or active compound derived therefrom.

99. The method of claim 98 wherein improving pregnancy outcome comprises, or is associated with, one or more of the following: increasing the pregnancy rate; increasing the rate of implantation of an embryo; improving the quality of an embryo; and improving the development of an embryo.

100. The method of claim 98 wherein the pregnancy is a naturally conceived pregnancy.

101. The method of claim 98 wherein the pregnancy is produced by an assisted reproduction technology.

102. The method of claim 101 wherein the assisted reproduction technology is selected from the group consisting of artificial insemination, in vitro fertilization, gamete intrafallopian transfer (GIFT), intra-uterine insemination (IUI), intracytoplasmic sperm injection (ICSI), testicular sperm extraction (TESE), and percutanenous epididymal sperm aspiration (PESA).

103. A composition for improving male reproductive health or improving pregnancy outcome, the composition comprising:
(i) an effective amount of at least one anti-oxidant agent;
(ii) an effective amount of at least one agent that reduces inflammation in the male reproductive tract and/or that increases testicular testosterone concentration; and
(iii) an effective amount of at least one agent that improves sperm function and/or is involved in cellular DNA synthesis,
wherein the at least one agent that reduces inflammation in the male reproductive tract and/or increases testicular testosterone concentration comprises garlic or an extract, oil or active compound derived therefrom.

104. A composition of claim 103, comprising Vitamin E; Vitamin C, or a salt thereof; lycopene; selenium; zinc; and at least about 500 mg garlic, or an extract or oil thereof; or a pharmaceutically acceptable derivative of any of the aforementioned components; the composition optionally further comprising folic acid, or a salt thereof, and/or Co Enzyme Q10.

105. The composition of claim 103 comprising about 0.5 to 50 mg lycopene, about 10 to 1000 mg Vitamin C, about 40 to 4000 I.U. Vitamin E, about 10 to 250 μg selenium, about 2.5 to 100 mg zinc, at least about 500 mg garlic, and optionally about 4 to 400 mg Co-Enzyme Q10.

106. The composition of claim 103 comprising about 2 to 10 mg lycopene, about 20 to 200 mg Vitamin C, about 200 to 600 I.U. Vitamin E, about 20 to 50 μg selenium, about 10 to 50 mg zinc, at least about 500 mg garlic, and optionally about 10 to 100 mg Co-Enzyme Q10.

107. The composition of claim 103 comprising garlic oil equivalent to about 1000 mg garlic bulb.

108. The composition of claim 103 comprising about 6 mg lycopene, about 100 mg Vitamin C, about 400 I.U. Vitamin E, about 26 μg selenium, about 25 mg zinc, about 1000 mg garlic, and optionally about 40 mg Co-Enzyme Q 10.

109. The composition of claim 103 further comprising about 500 μg folate.

110. A combination product comprising the following components:
at least one anti-oxidant agent;
at least one agent that reduces inflammation in the male reproductive tract; and/or that increases testicular testosterone concentration; and
at least one agent that improves sperm function and/or is involved in cellular DNA synthesis,
wherein the at least one agent that reduces inflammation in the male reproductive tract and/or increases testicular testosterone concentration comprises garlic or an extract, oil or active compound derived therefrom,
and wherein each of said components in the combination product are different, the components being provided as a plurality of individual dosage forms and wherein the combination product is provided with a set of instructions directing the administration of at least one of each individual dosage forms so as to improve male reproductive health or improve pregnancy outcome.

111. The combination product of claim 110 wherein the at least one anti-oxidant agent is selected from one or more of the group consisting of a β-carotenoid, comprising lycopene, lutein, and zeaxanthin; Vitamin C; Vitamin E; Co-Enzyme Q10; selenium; zinc; L-carnitine; acetylcarnitine; N-acetylcysteine; glutathionine; pyruvate; and hypotaurine.

112. The combination product of claim 110 wherein the at least one agent that reduces inflammation in the male reproductive tract further comprises one or more of the group consisting of green tea, or an extract or active compound derived therefrom; N-3 fatty acids, docosahexaenoic acid, ginger or an extract or active compound derived therefrom; an agent that decreases leukocyte production of TNFα, comprising pentoxyphylline; an agent that blocks the action of TNFα once produced, comprising infliximab; and lipid extract from marine mollusk.

113. The combination product of claim 110 wherein the at least one agent that increases testicular testosterone concentration further comprises one or more of the group consisting of zinc; tribulus terrestris or an extract or active compound derived therefrom;
an agent that reduces inducible nitric oxide synthase in macrophages; and an agent that reduces nitric oxide production.

114. The combination product of claim 110 wherein the administration to the subject comprises daily administration of about 0.5 to 50 mg lycopene, about 10 to 1000 mg Vitamin C, about 40 to 4000 I.U. Vitamin E, about 10 to 250 μg selenium, about 2.5 to 100 mg zinc, at least about 500 mg garlic, and optionally about 4 to 400 mg Co-Enzyme Q10.

115. The combination product of claim 110 wherein the administration to the subject comprises daily administration of about 2 to 10 mg lycopene, about 20 to 200 mg Vitamin C, about 200 to 600 I.U. Vitamin E, about 20 to 50 μg selenium, about 10 to 50 mg zinc, at least about 500 mg garlic, and optionally about 10 to 100 mg Co-Enzyme Q10.

116. The combination product of claim 110 wherein the administration to the subject comprises daily administration of garlic oil equivalent to about 1000 mg garlic bulb.

117. The combination product of claim 110 wherein the administration to the subject comprises daily administration of about 6 mg lycopene, about 100 mg Vitamin C, about 400 I.U. Vitamin E, about 26 μg selenium, about 25 mg zinc, about 1000 mg garlic, and optionally about 40 mg Co-Enzyme Q10.

118. The combination product of claim 110 wherein administration to a subject is for a period of at least 10 weeks.

119. The combination product of claim 110 wherein the at least one agent that improves sperm function and/or is involved in cellular DNA synthesis is folic acid or a salt thereof.

120. The combination product of claim 110 wherein the at least one agent that improves sperm function and/or is involved in cellular DNA synthesis is folic acid or a salt thereof.

121. The combination product of claim 110 wherein the least one of each individual dosage forms is administered daily.

122. The combination product of claim 110 wherein one or more of the at least one individual dosage forms is administered orally to the subject.

123. Semen or sperm isolated from a male subject treated according to the method of claim 84.

124. A method of isolating sperm from a male subject, the method comprising:
(i) administering to the male subject an effective amount of at least one anti-oxidant agent;
(ii) administering to the male subject an effective amount of at least one agent that reduces inflammation in the male reproductive tract and/or increases testicular testosterone concentration;
(iii) administering to the male subject an effective amount of at least one agent that improves sperm function and/or is involved in cellular DNA synthesis; and
(iv) isolating sperm from the male subject, wherein the at least one agent that reduces inflammation in the male reproductive tract and/or increases testicular testosterone concentration comprises garlic or an extract, oil or active compound derived therefrom.

125. Sperm isolated according to the method of claim 124.

126. A non-human animal arising from fertilization of a female non-human animal with sperm isolated according to the method of claim 124.
